(19) United States
(12) Patent Application Publication
(10) Pub. No.: US 2015/0104868 A1
(43) Pub. Date: Apr. 16, 2015

(54) Methods of Maintaining and Improving Biological Cell Function and Activity

(71) Applicant: GARGILL INCORPORATED, Wayzata, MN (US)

(72) Inventors: Alvin Berger, Long Lake, MN (US); Aalt Bast, Nijkerk (NL); Petrus Wilhelmus Hubertus De Cock, Keerbergen (BE); Gerardus Johannes Martinus Den Hartog, Maastricht (NL); Jean-Claude Marie-Pierre Ghislain De Troostembergh, Houwaart (Tielt-Winge) (BE)

(73) Assignee: CARGILL, INC., Wayzata, MN (US)

(21) Appl. No.: 14/396,046

(22) PCT Filed: Apr. 19, 2013
(86) PCT No.: PCT/US2013/037342

§ 371 Date: Oct. 21, 2014

Related U.S. Application Data
(60) Provisional application No. 61/636,673, filed on Apr. 22, 2012.

(51) Int. Cl. C12N 5/071; C12N 1/38

(52) U.S. Cl. CPC C12N 5/069; C12N 5/0679; C12N 1/38; C12N 5/0676; C12N 2501/999
USPC 435/366; 435/375; 435/420; 435/244; 435/255.1; 435/254.1; 435/252.1; 435/377

(57) ABSTRACT

The present invention relates to methods of using meso-1,2,3,4-tetrahydroxybutane for the maintenance and/or improvement of biological cell function and activity, and for the prevention of improper cell functioning or cell death, in vitro, ex vivo, and in vivo over time and/or during exposure to stress. Meso-1,2,3,4-tetrahydroxybutane can be used to promote cell survival and as a cell protection agent, to increase cell viability, and to improve conversion of progenitor or stem cells to mature cells, whether in vitro, in vivo, ex-vivo, or transplanted.

### TECHNICAL FIELD

The present invention relates to methods of using meso-1,2,3,4-tetrahydroxybutane for the maintenance and/or improvement of biological cell function and activity, and for the prevention of improper cell functioning or cell death, in vitro, ex vivo and in vivo over time and/or during exposure to stress.

### BACKGROUND

Cells exposed to stress caused from such events including, but not limited to, exposure to glucose or other sugars or substances formed or elevated by their presence that can cause stress, do not maintain their normal activity and function in vitro, ex vivo, and in vivo, and may even die. High cell death is a problem in vitro and ex vivo, when cells, such as, for example, commercially important cells from animals, humans, plants, bacteria, yeast and fungi, are exposed to high cell densities and high glucose or sugar media conditions. High cell death is also a problem in viva, when cells within organs are exposed to high glucose or sugar and other stressors formed or elevated by the presence of glucose or sugar. In vivo cell death or improper cell functioning occurs particularly in pre-disease or disease states including, for example, pre-diabetes, diabetes (Types I and II), metabolic syndrome, chronic inflammatory diseases such as Alzheimer's disease, prostate cancer, breast cancer, atherosclerosis, colon cancer, and cardiovascular diseases in general. Thus, there is an on-going need to find an agent or therapy to prevent and/or mitigate such improper cell functioning and cell death, in vitro, ex vivo, and in vivo.

Of the above-mentioned diseases, diabetes is of particular interest because of its growing prevalence. Diabetes is a general term for diseases characterized by excessive urination. The most studied form of diabetes is diabetes mellitus (DM), which is a chronic disorder of carbohydrate metabolism characterized by hyperglycemia (blood glucose >8 mM) and glucosuria (presence of glucose in the urine), and resulting from inadequate production or use of insulin. DM is classified according to two syndromes: Type I, or insulin-dependent DM (IDDM), and Type II, or non-insulin-dependent DM (NIDDM). In Type I, the patient secretes little or no insulin. In Type II, insulin is produced but exogenous insulin or blood sugar lowering drugs are needed to control hyperglycemia, because the patient is unable to detect or process insulin on his/her own. Type II DM occurs much more frequently than Type I.

In addition, a number of important and meticulously performed epidemiological studies have highlighted the relationship between hyperglycemia and an increased risk of cardiovascular diseases. These cardiovascular diseases include microvascular pathologies in the eye (retina), kidney, and peripheral nerves. As a consequence, DM is a leading cause of blindness, renal disease, and a variety of debilitating neuropathies. DM is also associated with the formation of atherosclerotic lesions in arteries that supply the heart, brain, and limbs. As a result, patients with DM have a much higher risk of myocardial infarction, stroke, and amputation. Taken together, these consequences of chronic hyperglycemia are referred to as diabetic complications, which in turn result in a high level of stress on the cells.

A need still exists for the development of methods to maintain and/or improve biological function and activity of cells over time and/or during exposure to stress, such as cell stress related to various conditions and diseases associated with chronic hyperglycemia in diabetes, including microvascular pathologies in the retina, kidney and peripheral nerves. Further, given its ability of high availability in cells, there is a need to discover novel biological benefits and associated mechanisms by which cells can be protected by the compound meso-1,2,3,4-tetrahydroxybutane and its formulations.

### SUMMARY

The disclosure relates to novel methods of using meso-1,2,3,4-tetrahydroxybutane, and formulations comprising the compound, to maintain and/or improve biological function and activity of cells over time and/or during exposure to stress. The disclosure also relates to novel methods of preventing or treating chronic inflammatory or aging-related diseases such as Alzheimer's disease, prostate cancer, breast cancer, atherosclerosis, colon cancer, diabetes, and cardiovascular diseases using the compound of meso-1,2,3,4-tetrahydroxybutane and its formulations.

Disclosed herein is the use of meso-1,2,3,4-tetrahydroxybutane, in pure or substantially pure form, or formulated in a product or composition, to maintain or improve biological function and activity of cells over time and/or during exposure to stress, whereby cells include cells from animals, humans, and plants, as well as bacteria, yeasts, and fungi. Also disclosed herein is the use of meso-1,2,3,4-tetrahydroxybutane, in pure or substantially pure form, or formulated in a product or composition, as a cell survival and cell protection agent, to increase cell viability, whereby cells include cells from animals, humans, plants, as well as mircoorganisms such as bacteria, yeasts, and fungi. Also disclosed herein is the use of meso-1,2,3,4-tetrahydroxybutane, in pure or substantially pure form, or formulated in a product or composition, to improve conversion of progenitor or stem cells to mature cells, whether in vitro, in vivo, ex-vivo, or transplanted.

Disclosed herein is the use of meso-1,2,3,4-tetrahydroxybutane as described above, whereby cells are exposed to stress conditions caused directly or indirectly by glucose or other monosaccharides and disaccharides. In certain embodiments, the cells may be progenitor and mature cells lining up the inside of the vascular system. In certain embodiments disclosed herein, the cells may be progenitor and mature beta cells located in the islets of Langerhans.

In certain embodiments, the cells may be progenitor and mature brain cells, including but not limited to all cells inside the cranium or in the central spinal canal, in lymphatic tissue, in blood vessels, in the cranial nerves, in the brain envelopes (meninges), skull, pituitary gland, and/or pineal gland, and within the brain itself the cells may be neurons and/or glial cells (which include astrocytes, oligodendrocytes, and ependymal cells).

In certain embodiments, the cells may be microbial intestinal cells, as well as progenitor and mature host intestinal cells, including but not limited to cells in the colon, rectum, or in the appendix. In certain embodiments, the cells may be progentor and mature prostate cells or progenitor and mature breast cells, including but not limited to epithelial cells from the ducti and lobules, and adipocytes. In certain other embodiments, the cells may be microorganisms used in the fermentation step of the production process of food products including but not limited to fermented milk or milk derivatives, yogurt, cheese, bread, pastry, sauces, pastes, wine, beer, sauerkraut, kimchi, tempeh, bean paste, etc. In certain embodiments, the cells may be microorganisms used as production strain for the manufacture of substances that are used as food, food ingredients and additives, nutraceuticals, pharmaceuticals, and/or personal care products.

There are many benefits and indications of use of the compound of meso-1,2,3,4-tetrahydroxybutane, and formulations comprising mesa-1,2,3,4-tetrahydroxybutane, including but not limited to: (1) helping cells survive and maintain function over time and/or under stress conditions, as well as reduce and/or retard the aging process of cells; (2) reducing the risk of diseases, including but not limited to, pre-diabetes, diabetes (Types I and II), metabolic syndrome, chronic inflammatory diseases such as Alzheimer's disease, prostate cancer, breast cancer, atherosclerosis, colon cancer, diabetes, and cardiovascular diseases in general; (3) improving beta cell function and insulin resistance, which may be important for treating large populations with impaired glucose metabolism, such as Type I or Type II diabetes, or individuals with the metabolic syndrome; (4) improving cell transplantation and progenitor (stem) cell survival with broad economic and biomedical importance; (5) enhancing cell survival and promoting activity of microorganisms used in fermentation processes (such as probiotic cultures, cultures used in dairy, yeasts used in brewery and baking, cells producing clinically important products such as proteins, microorganisms used to produce substances used as foods, food ingredients, pharmaceuticals, personal care products, etc.); (6) to help microorganisms survive and maintain function and activity during preservation processes such as, for example, concentration, drying, freezing, and upon storage; and (7) in the food industry, particularly the industry involving probiotic survival and functionality among ingredient and food companies and clinicians, to induce small increases in production-yield of commercially-important cells that are of high economic importance to bioprocessors.

### BRIEF DESCRIPTION OF THE FIGURES

FIG. 1 is a graph illustrating a significant increase in the amount of dead cells after the HUVECs incubation with HG (30 mM) for 24 hours. In FIG. 1, n equals 4, # equals significantly different compared to blank (p<0.05), and * equals significantly different compared to high glucose exposure (p<0.05);

FIG. 2 is a graph illustrating a significant increase in the amount of dead cells after the HUVECs incubation with Sin-1 for 24 hours. In FIG. 2, n equals 3, and # equals significantly different compared to blank (p<0.05);

FIG. 3 is a graph illustrating a significant increase in the amount of dead cells after the HUVECs incubation with CML for 24 hours. In FIG. 3, n equals 3, and # equals significantly different compared to blank (p<0.05), and * equals significantly different compared to Nε-(carboxymethyl)lysine exposure (p<0.05);

FIGS. 4A-4C provide a set of graphs illustrating the effects of meso-1,2,3,4-tetrahydroxybutane, decreasing the percentage of dead HUVEC cells (with the y-axis representing the percentage of dead cells);

FIG. 5 is a graph illustrating the cell death at different concentrations of sugars (with the y-axis representing the leakage of lactate dehydrogenase in U/L);

FIG. 6 is a graph illustrating the effects of incubation with increasing levels of glucose on viability (function and activity) of HIT-T15 cells;

FIG. 7A is a graph showing the effect on viability of 1.1E7 cells incubated with 10, 30 and 45 mM glucose for 24 hours;

FIG. 7B is a graph showing the effect on viability of 1.1E7 cells incubated with 10, 30 and 45 mM glucose for 48 hours;

FIG. 7C is a graph showing the effect on viability of 1.1E7 cells incubated with 10, 30 and 45 mM glucose for 6 days. In FIG. 7C, * equals p<0.05 compared to 10 mM (normal glucose), and ** equals p<0.05 compared to no meso-1,2,3,4-tetrahydroxybutane;

FIG. 7D is a graph showing the effect on viability of 1.1E7 cells incubated with 10, 30 and 45 mM glucose for 7 days. In FIG. 7D, * equals p<0.05 compared to 10 mM (normal glucose), and ** equals p<0.05 compared to no meso-1,2,3,4-tetrahydroxybutane;

FIG. 8A is a graph showing the effect on gene expression of 7B2 of 1.1E7 cells incubated with 10, 30 and 45 mM glucose for 24 hours. In FIG. 8A, * equals p<0.05 compared to 10 mM (normal glucose), and ** equals p<0.05 compared to no meso-1,2,3,4-tetrahydroxybutane;

FIG. 8B is a graph showing the effect on gene expression of 7B2 of 1.1E7 cells incubated with 10, 30 and 45 mM glucose for 7 days. In FIG. 8B, * equals p<0.05 compared to 10 mM (normal glucose);

FIG. 8C is a graph showing the effect on gene expression of PC1/3 of 1.1E7 cells incubated with 10, 30 and 45 nM glucose for 24 hours. In FIG. 8C, ** equals p<0.05 compared to no meso-1,2,3,4-tetrahydroxybutane;

FIG. 8D is a graph showing the effect on gene expression of PC1/3 of 1.1E7 cells incubated with 10, 30 and 45 nM glucose for 7 days. In FIG. 8D, * equals p<0.05 compared to 10 mM (normal glucose);

FIG. 8E is a graph showing the effect on gene expression of PC2 of 1.1E7 cells incubated with 10, 30 and 45 nM glucose for 24 hours. In FIG. 8E, ** equals p<0.05 compared to no meso-1,2,3,4-tetrahydroxybutane;

FIG. 8F is a graph showing the effect on gene expression of PC2 of 1.1E7 cells incubated with 10, 30 and 45 nM glucose for 7 days. In FIG. 8F, * equals p<0.05 compared to 10 mM (normal glucose);

FIG. 9 is a graph showing blood glucose levels before and after meso-1,2,3,4-tetrahydroxybutane intake (n=24);

FIG. 10 is a graph showing blood insulin levels before and after meso-1,2,3,4-tetrahydroxybutane intake (n=24); and

FIG. 11 is a graph showing blood C-peptide levels before and after meso-1,2,3,4-tetrahydroxybutane intake (n=24).

### DETAILED DESCRIPTION

The disclosure relates, in exemplary embodiments, to methods of using meso-1,2,3,4-tetrahydroxybutane, and formulations comprising meso-1,2,3,4-tetrahydroxybutane, for the maintenance or improvement of biological cell function and activity, over time and/or as the cells are exposed to stress conditions in vivo, ex vivo, or in vitro.

Meso-1,2,3,4-tetrahydroxybutane (“ERT”) has been prepared as a white, anhydrous, non-hygroscopic, crystalline substance, available in powdered or granular form with a mild sweetness (about 60-70% as sweet as sucrose) and appearance similar to sucrose. Its small molecular size is responsible for many of ERT's unique characteristics. Due to its small molecular size, about 90% of the ingested ERT is absorbed in the small intestine. While it is well-absorbed, it is not metabolized. The kidneys remove ERT from the bloodstream and it is excreted unchanged in the urine. The small amount of remaining ERT that is not absorbed passes into the large intestine and is excreted unchanged in the feces. As ERT is not metabolized or fermented in the colon, it is non-caloric and very well-tolerated. ERT contributes no energy at all to the body. Since ERT is not metabolized, it does not have any glycemic or insulinemic effect. This makes it a particularly useful for people wishing to reduce their post-prandial blood sugar levels.

ERT occurs naturally in a wide variety of fruits, vegetables and fermented foods. It is also present in the human body and in animals. ERT is produced by a natural fermentation process. ERT does not promote tooth decay as it cannot be used or is only poorly used as a substrate by oral bacteria that cause dental caries such as Streptococcus mutans. ERT can reduce dental plaque, thereby reducing the risk of developing dental caries.

ERT is used as a food item (in its pure form) and as an ingredient or additive in many food and beverages, in pharmaceuticals, and in personal care products. In table-top sweetener applications, ERT is used “as-is” (in its pure form as it is sold on the market) without the addition of any other ingredients, or at levels up to 99.9% as a non-caloric, non-cariogenic, non-glycemic carrier for intense sweeteners. In these applications, the sensorial profile-modifying properties of ERT are of great importance resulting in sweetness synergy, improved mouth-feel, and masking of off-flavors. In addition, due to ERT's crystalline structure and density similar to sucrose, and its non-hygroscopic property, it offers excellent flowability and stability as carrier.

The quantitative and qualitative synergies that ERT shows in combination with intense sweeteners are also very useful in low-calorie and diet beverages. ERT is often used as a flavor enhancer in drinks to achieve a sweetness profile that comes close to that of regular sugar. Good quality non-caloric and non-cariogenic chewing gum can also be formulated using ERT. The use of ERT in chocolate compositions allows a dry couching process at high temperatures. Due to the good heat stability and low-moisture pick-up of ERT, it is even possible to work at higher temperatures than traditionally used. This results in an enhanced flavor development. For example, sugarfree fudge with texture and shelf-life properties equivalent to conventionally sweetened fudge can be produced using ERT.

The latest candy innovation involves technology that broadens the melting as well as the crystallization peak of ERT and shifts the melt crystallization peak to a level low enough to allow depositing in moulds and control the formation of crystals. Resulting hard candies have a smooth appearance, texture, and desired hardness. This novel technology allows the manufacture of deposited candies that are essentially free of calories, are safe for teeth, and have a novel texture and taste sensation.

Using ERT, it is possible to obtain sugarfree, low calorie, noncariogenic fondant, lozenges, tablets and many other types of candies with similar properties to classical sugar-containing products. In bakery product applications, ERT generally gives a somewhat different melting behavior, more compact dough, softer end products, and less color formation compared to sucrose.

In pharmaceutical and personal care applications, ERT is used, for example, for its technical functionality, dental health benefits, and for its metabolic characteristics and high inertness.

The current disclosure provides new and previously unknown benefits of ERT, namely its ability to maintain and/or improve biological function and activity of cells over time and/or during exposure to stress.

Previous research has shown that the use of meso-1,2,3,4-tetrahydroxybutane in vitro was effective in scavenging hydroxyl radicals (scavenges HO• radicals), but does not possess any superoxide or peroxynitrite scavenging activity. Additionally, in an in vitro model of radical-induced hemolysis, meso-1,2,3,4-tetrahydroxybutane was able to delay the onset of oxidative damage, in particular ABTS-induced hemolysis.

Additional research and results surrounding the use of related compounds of meso-1,2,3,4-tetrahydroxybutane for the prevention or treatment of hypertension are described in the International Publication No. WO 2011/014448 A1, which is herein incorporated by reference in its entirety.

New studies are disclosed herein, which demonstrate novel methods of using the compound of meso-1,2,3,4-tetrahydroxybutane and formulations comprising meso-1,2,3,4-tetrahydroxybutane to protect, maintain, and improve biological cell function. The effects of using meso-1,2,3,4-tetrahydroxybutane in HUVEC cells were further studied as the cells were exposed to normal and high glucose conditions, using targeted and transcriptomic approaches. These studies demonstrate that meso-1,2,3,4-tetrahydroxybutane reduces high glucose-induced cell death. Changes to gene transcripts in various pathways, described in detail below, may clarify how meso-1,2,3,4-tetrahydroxybutane exerts beneficial effects, including how the use of the compound of meso-1,2,3,4-tetrahydroxybutane and formulations comprising meso-1,2,3,4-tetrahydroxybutane, alone or in combination therapy, may reduce diabetic complications relating to cell damage and/or death.

### DEFINITIONS

The terms “disease,” “condition,” and the like, as used herein, are intended to mean any deviation or interruption of the normal structure or function of any part, organ, or system, or combination thereof of an animal or human, that is manifested by a characterisitc set of symptoms and signs and whose etiology, pathology, and prognosis may be known or unknown. The term “animal,” as used herein, includes mammals, including but not limited to humans and members of the equine, porcine, bovine, murine, canine or feline species, for example.

The term “hyperglycemia,” as used herein, refers to a condition in which an excessive amount of glucose is present in the blood serum. The physiological blood glucose level is typically about 5 to 7 mmol/l. A blood sugar level of 10 mmol/l (or chronically above 7 mmol/l) or more is typically considered hyperglycemic.

The term “prevention” as used herein includes (i) any activity which avoids the development of a disease of an animal or human which may be predisposed to the disease but has not yet been diagnosed as having it, and (ii) any activity which is aimed at early detection, thereby increasing opportunities for intervention to prevent progression of the disease and emergence of symptoms. Further, the term “treatment” as used herein refers to, in any degree: (i) any activity which protects against a disease by inhibiting the disease, i.e., arrests the disease development, and (ii) any activity which relieves the disease, i.e., causes regression or disappearance of the disease.

The term “biological function” as used herein for cells refers to the ability to contribute to sustaining and/or replicating in a controlled fashion. In particular, it may include a timely and adequate production of molecules that are important to stay alive, and/or important for the cell to provide the tissue of which it is part of the desired functions and mechanical properties, and/or important to be used extracellularly, and it may include the property of the cellular system to regulate its internal environment to maintain a stable and relatively constant condition of properties.

The use of meso-1,2,3,4-tetrahydroxybutane can maintain or improve biological cell function and activity and prevent improper cell functioning or cell death, in vitro, ex vivo, and in vivo, for cells exposed to stress. Additionally, the use of meso-1,2,3,4-tetrahydroxybutane can promote cell survival and act as a cell protection agent that can increase cell viability. The use of meso-1,2,3,4-tetrahydroxybutane can also improve conversion of progenitor or stem cells to mature cells, either in vitro, in vivo, ex-vivo, or transplanted. The affected cells include those from animals, humans, plants, and microorganisms including bacteria, yeasts and fungi.

The meso-1,2,3,4-tetrahydroxybutane compound may be used, in at least certain non-limiting exemplary embodiments, in a pure, or substantially pure, form and comprise greater than, by way of example only, 99% mesa-1,2,3,4-tetrahydroxybutane and 1% water or less, 99.5% meso-1,2,3,4-tetrahydroxybutane and 0.5% water or less, or 99.9% meso-1,2,3,4-tetrahydroxybutane and 0.1% water or less, or may be formulated into a product or composition. A composition comprising meso-1,2,3,4-tetrahydroxybutane may, for example, comprise less than 20% meso-1,2,3,4-tetrahydroxybutane when used or consumed in relatively large amounts, 20% to 50% meso-1,2,3,4-tetrahydroxybutane when used or consumed in greater amounts, and greater than 50% meso-1,2,3,4-tetrahydroxybutane when used or consumed in relatively low amounts.

The pure, or substantially pure, form of the meso-1,2,3,4-tetrahydroxybutane compound or its formulations may be administered by various routes known to those of skill in the art. The route of administration is not particularly limited, and is determined by the preparation form, and the condition of the animal or human to be prevented or treated, such as, for example, age, sex and the degree of disease or condition. For example, the pure or substantially pure meso-1,2,3,4-tetrahydroxybutane compound, or compositions or formulations comprising meso-1,2,3,4-tetrahydroxybutane, can be administered orally, parenterally, subcutaneously, intravenously, intramuscularly, by inhalation spray, topically, rectally, nasally, buccally, vaginally, or transdermally. In various embodiments, pure meso-1,2,3,4-tetrahydroxybutane, or formulations or compositions comprising mesa-1,2,3,4-tetrahydroxybutane, may also be easily incorporated into food products, beverage products, or other orally used products.

The meso-1,2,3,4-tetrahydroxybutane may be formulated as, for example, a pharmaceutical product or composition for human or animal consumption, including a chemical product or composition capable of inducing a desired therapeutic effect, when administered to a patient, and which is formulated by mixing meso-1,2,3,4-tetrahydroxybutane, and optionally other active ingredients, with one or more well-known substances such as physiologically acceptable carriers, diluents, and other agents that are usually incorporated into pharmaceutical formulations to provide improved transfer, delivery, tolerance, and the like. In addition, compounds such as antioxidants, dispersants, emulsifiers, flavorings, sweeting agents, coloring agents, and preservatives may also optionally be included in the product or composition.

Suitable forms of the pharmaceutical product or composition of the present invention include, for example, solid forms, such as powders, tablets, pills, capsules, cachets, suppositories and granules, and liquid forms, such as solutions, syrups, suspensions and emulsions. The appropriate form of the pharmaceutical product or composition is primarily guided by the route of administration, the desired release profile, and other factors such as incompatibilities of active substance and pharmaceutical excipients. A person skilled in the art of pharmaceutical formulations is able to choose in routine fashion the form and preparation method with reference to known material and process parameters. For oral administration, for example, tablets may contain carriers, such as, but not limited to, ERT, lactose and corn starch, and/or lubricating agents, such as magnesium stearate. Further, capsules may contain diluents including, but not limited to, ERT, lactose and dried corn starch. Moreover, aqueous solutions or suspensions may contain emulsifying and suspending agents.

Previous studies have found that incorporating mesa-1,2,3,4-tetrahydroxybutane by itself (i.e., under non-diabetic conditions) has minimal effects on cells. Viability, oxidative damage, superoxide dismutase activity, and important cell function parameters (nitric oxide production, adhesion molecule production) and the transcriptome did not show changes after incubation with meso-1,2,3,4-tetrahydroxybutane. However, when cells were exposed to high glucose following pre-incubation with meso-1,2,3,4-tetrahydroxybutane, surprisingly a number of the deleterious effects caused by the high glucose stress condition were reversed.

Furthermore, it was found that in non-diabetic subjects, meso-1,2,3,4-tetrahydroxybutane did not affect cells, which is a desirable property, while in diabetic subjects where the cells are under diabetic stress, meso-1,2,3,4-tetrahydroxybutane surprisingly could shift a variety of damage and dysfunction parameters to a safer and more tolerable degree. Meso-1,2,3,4-tetrahydroxybutane has been shown as having definite protective effects on cells during hyperglycemia, that may assist other safe agents in reducing the risk of developing diabetic complications. Meso-1,2,3,4-tetrahydroxybutane is therefore of great importance to a rapidly growing population of people with diabetes in reducing their risk of developing diabetic complications.

The following Examples are provided to illustrate the use of meso-1,2,3,4-tetrahydroxybutane in the maintenance and improvement of biological cell function and activity or cells as they age or as they are exposed to stress conditions. It is to be understood that both the foregoing description and the following Examples are exemplary and explanatory only, and are not to be interpreted as restrictive of the disclosure, with the scope of the invention being defined by the claims. Moreover, it should be understood that various features and/or characteristics of differing embodiments herein may be combined with one another. It is therefore to be understood that numerous modifications may be made to the illustrative embodiments and that other arrangements may be devised without departing from the true spirit and scope of the invention. Other embodiments will be apparent to those skilled in the art from consideration of the disclosure and practice of the various exemplary embodiments disclosed herein.

It is also to be understood that, as used herein the terms “the,” “a,” or “an,” mean “at least one,” and should not be limited to “only one” unless explicitly indicated to the contrary.

Unless otherwise indicated, all numbers used in the specification and claims are to be understood as being modified in all instances by the term “about,” whether or not so stated. It should also be understood that the precise numerical values used in the specification and claims form additional embodiments of the invention, and are intended to include any ranges which can be narrowed to any two end points disclosed within the exemplary ranges and values provided. Efforts have been made to ensure the accuracy of the numerical values disclosed herein. Any measured numerical value, however, can inherently contain certain errors resulting from the standard deviation found in its respective measuring technique.

### EXAMPLES

For the following Example 1, the parameters that were evaluated included (i) general toxicity (targeted) evaluated by viability and appearance; (ii) oxidative damage (targeted) evaluated by lipid peroxidation, protein carbonyl formation, and oxidized nucleotide formation; (iii) cell function (targeted) evaluated by NO production, expression of pro- and anti-inflammatory genes (Polymerase Chain Reaction (PCR)), and adhesion molecule production; (iv) eicosanoidomics; and (v) transcriptomics.

The following abbreviations are used in the Examples:

| | |
|---|---|
| AGE | Advanced glycation end product |
| ATCC | American Type Culture Collection |
| CML | Nε-(carboxymethyl)lysine |
| CYP | Cytochrome P 450 oxidase |
| DM | Diabetes mellitus |
| DNA | Deoxyribonucleic acid |
| ERK | Extracellular receptor regulated kinase |
| ERT | meso-1,2,3,4-tetrahydroxybutane |
| FCS | Fetal calf serum |
| GCC | Geotrichum candidum cells |
| HBSS | Hank's Balanced Salt Solution |
| 12-HETE | 12-Hydroxyeicosatetraenoic acid |
| 12(S)-HETE | S enantiomer of 12-hydroxyeicosatetraenoic acid |
| HG | High glucose or hypergycemic |
| HGCML | High glucose plus Nε-(carboxymethyl)lysine |
| HGCMLERT | High glucose plus Nε-(carboxymethyl)lysine plus meso- |
| | 1,2,3,4-tetrahydroxybutane |
| HGCMLMAN | High glucose plus Nε-(carboxymethyl)lysine plus |
| | (2R,3R,4R,5R)-hexaan-1,2,3,4,5,6-hexol |
| HGERT | high glucose plus meso-1,2,3,4-tetrahydroxybutane |
| HGMAN | high glucose plus (2R,3R,4R,5R)-hexaan-1,2,3,4,5,6-hexol |
| HGSIN | high glucose plus 1,3-morpholino sidnonimine |
| HGSINERT | high glucose plus 1,3-morpholino sidnonimine plus meso- |
| | 1,2,3,4-tetrahydroxybutane |
| HGSINMAN | high glucose plus 1,3-morpholino sidnonimine plus |
| | (2R,3R,4R,5R)-hexaan-1,2,3,4,5,6-hexol |
| HUVEC | Human umbilical vein endothelial cells |
| IDDM | Insulin-dependent diabetes mellitus |
| IG | Intermediate glucose |
| JNK | c-Jun N-terminal kinase |
| MAN | (2R,3R,4R,5R)-hexaan-1,2,3,4,5,6-hexol |
| mM | Millimolar millimoles per liter |
| NG | Normal glucose or normoglycemic |
| NGCML | Normal glucose plus Nε-(carboxymethyl)lysine |
| NGCMLERT | Normal glucose plus Nε-(carboxymethyl)lysine plus meso- |
| | 1,2,3,4-tetrahydroxybutane |
| NGCMLMAN | Normal glucose plus Nε-(carboxymethyl)lysine plus |
| | (2R,3R,4R,5R)-hexaan-1,2,3,4,5,6-hexol |
| NGERT | Normal glucose plus meso-1,2,3,4-tetrahydroxybutane |
| NGMAN | Normal glucose plus (2R,3R,4R,5R)-hexaan-1,2,3,4,5,6- |
| | hexol |
| NGSIN | normal glucose plus 1,3-morpholino sidnonimine |
| NGSINERT | normal glucose plus 1,3-morpholino sidnonimine plus |
| | mesXB2o-1,2,3,4-tetrahydroxybutane |
| NGSINMAN | normal glucose plus 1,3-morpholino sidnonimine plus |
| | (2R,3R,4R,5R)-hexaan-1,2,3,4,5,6-hexol |
| NIDDM | Non-insulin-dependent diabetes mellitus |
| NO | Nitric oxide |
| P38 MAPK | P38 mitrogen activated protein kinase(s) |
| PC1 | Proprotein convertase 1 |
| PC2 | Proprotein convertase 2 |
| PKC | Protein kinase C |
| ROS | Reactice oxygen species |
| SAP | Stress activated protein |
| SIN-1 | 3-morpholinosidnonimine |
| SOD | Superoxide dismutase |
| TNFα | Tumor necrosis factor alpha |
| TXB2 | Thromboxane B2 |

### Example 1

### HUVEC Cells

A HUVEC cell line (CRL-1730) was obtained from American Type Culture Collection (ATCC) of Manassas, Va. The HUVECs were cultured in F12K medium (from ATCC) with 10% non-heat inactivated fetal calf serum (FCS, ATCC), 1% penicillin/streptomycin (Invitrogen), 0.05 mg/ml HUVEC growth supplements (ECGS, R&D systems) and 0.1 mg/mL heparin (Leo Pharmaceuticals). The prepared cells were maintained in collagen coated T75 flasks (Greiner Bio-one) at 37° C. in a 5% CO₂ atmosphere.

The prepared cells were subjected to various stressors. Stressors, for purposes of the examples, include conditions of stress caused by glucose directly or indirectly through the formation or elevation of other substances that cause stress. Diabetic stressors include high glucose (HG; 30 mM, 24 h), TNFα: pro-inflammatory agent, Nε carboxymethyllysine: advanced glycation end (AGE) product, and SIN-1: peroxynitrite generator. Prepared cells were also exposed to normal glucose (NG; 7 mM, 24 h) as a control.

The prepared cells were seeded in 6 well plates and T75 flasks and grown until 80% confluence. The medium was removed, and the cells were washed with Hank's Balanced Salt Solution (HBSS). A new medium without supplements was added and 1/10 volume of a 50 mM meso-1,2,3,4-tetrahydroxybutane (ERT) or (2R,3R,4R,5R)-hexaan-1,2,3,4,5,6-hexol (MAN) solution or vehicle solution was to added to the cells (5 mM final concentration). The vehicle solution is the solvent for the test compounds, in this case being the medium without supplements. After about 1 hour incubation, 1/10 volume of a 230 mM glucose solution (final concentration 30 mM) or the vehicle solution described above was added to the cells under a high glucose (HG) condition. The cells described above were also incubated for 24 hours with a 7 mM, normal glucose (NG) solution or in some instances, with a 15 mM, intermediary glucose (IG) solution. See, for example, FIG. 6. Subsequently, the cells were incubated for 24 hours. The same protocol was used for incubation with Nε-(carboxymethyl)lysine (CML, final concentration 1 micromolar) and 1,3-morpholino sidnonimine (SIN-1, final concentration 0.5 mM). Tumor necrosis factor alpha (TNFα, final concentration 0.1 ng/ml) was incubated for 3 hours.

Cell Viability

In a cell viability assay, fresh, untreated HUVEC cells were grown in 6 well plates until 80% confluence. Subsequently, the incubation medium was removed and the cells were washed with HBSS and harvested with trypsin. All cell material, including the medium and the HBSS wash, was collected and centrifuged for 5 minutes at 500×g. The supernatant was removed and the cells were re-suspended in 100 microliters of HBSS. The cell suspensions were diluted 1:1 in a 0.4% trypan blue dye (from Invitrogen) and loaded in a Burker counting chamber. The number of viable (white) and dead (blue) cells were counted in 25 squares, and the count was repeated an additional two times for each sample, for a total of three counts per sample. The average number was multiplied by 20,000, which represents the amount of cells per mL. The percentage of dead cells was then calculated with the formula: (blue cells/(blue cells+white cells))*100.

The results and data were collected. The preincubation with 5 mM ERT or 5 mM MAN followed by 24 hours of NG concentration did not result in increased cell death (NG vs NGERT; NG vs NGMAN). 5 mM indicates the final concentration, i.e., 1:10 of 50 mM. When the HUVECs were incubated with HG alone for 24 hours, the amount of dead cells increased almost 4-fold relative to the NG condition (NG vs HG). The preincubation with 5 mM ERT (HG vs HGERT) but not MAN(NG vs NGMAN), completely prevented the increase in the number of dead cells in the HG condition.

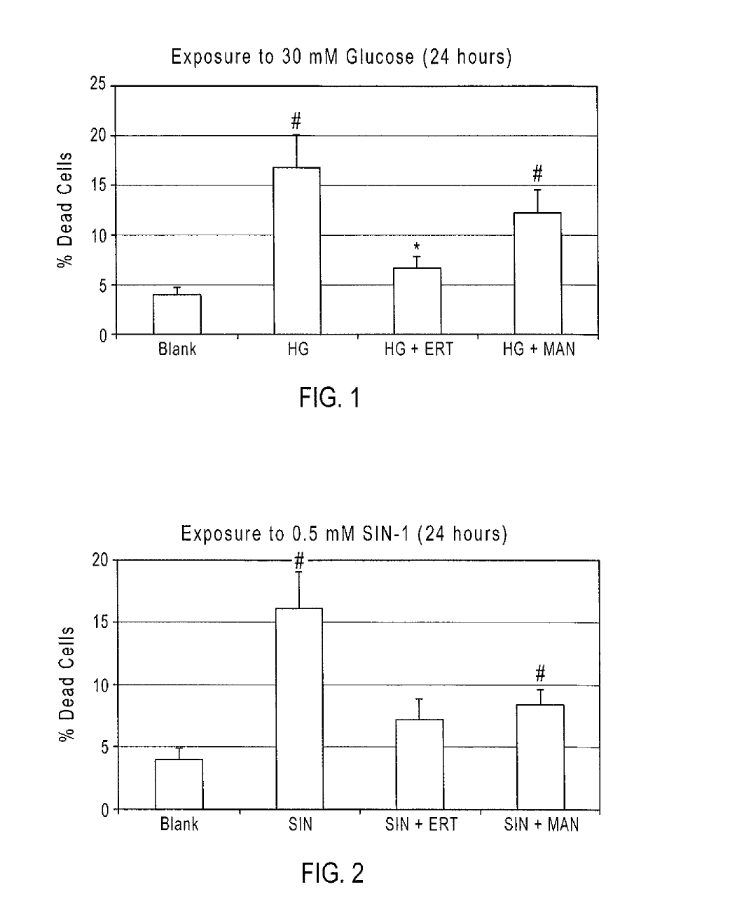

FIG. 1 illustrates how the HUVECs incubation with HG (30 mM) for 24 hours led to a significant increase in the amount of dead cells. The co-incubation with ERT (=E) significantly decreased this number, while the co-incubation with MAN (=M) did not have a significant effect of decreased amount of dead cells. In FIG. 1, the key includes: (n=4) # significantly different compared to blank (p<0.05); *=significantly different compared to high glucose exposure (p<0.05) meso-1,2,3,4-tetrahydroxybutane.

FIG. 2 illustrates that the HUVECs incubation with SIN-1 for 24 hours led to a significant increase in the amount of dead cells. The co-incubation with ERT (=E) decreased this number, while the co-incubation with MAN (=M) did not have this effect. The key for FIG. 2 includes: (n=3) #=significantly different compared to blank (p<0.05).

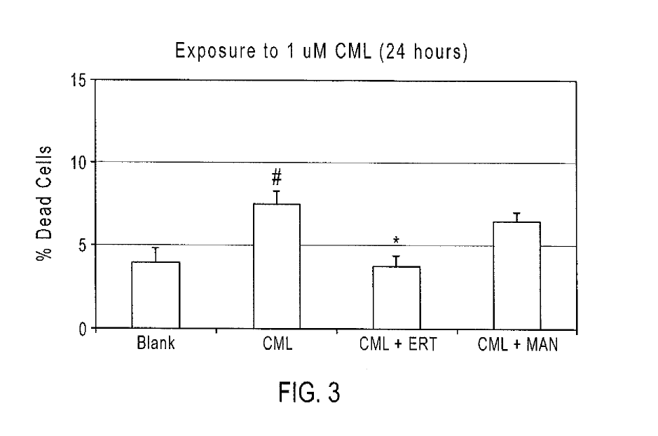

FIG. 3 illustrates that HUVECs incubation with CML for 24 hours led to a significant increase in the amount of dead cells. The co-incubation with ERT (=E) decreased this number, while the co-incubation with MAN (=M) did not have this effect. The key for FIG. 3 includes: (n=3) #=significantly different compared to blank (p<0.05), and * equals significantly different compared to Nε-(carboxymethyl)lysine exposure (p<0.05).

Oxidative Damage

Hyperglycemia is strongly associated with oxidative stress, an imbalance between the formation of reactive oxygen species and the present antioxidants. Excess reactive oxygen species react with critical cellular targets, viz. membrane lipids, proteins and (deoxy)ribonucleic acids. The effect of pre-incubation with meso-1,2,3,4-tetrahydroxybutane on the formation of oxidized lipids and proteins caused by high glucose was investigated to determine whether meso-1,2,3,4-tetrahydroxybutane could provide protection. Accordingly, one of the targeted assays included studying a comparison of HG to HGERT conditions, where the oxidative damage was evaluated showing that the protein carbonyls decreased from 6.55 nM/mg protein with HG to 3.77 with HGERT (p=0.089). Protein carbonyls are end products of reactions between proteins and reactive oxygen species (ROS), formed during oxidative stress. Protein carbonyls decreased from 6.55 nM/mg protein with HG to 3.77 with HGERT (p=0.089). As protein carbonyls and ERT are both hydrophilic molecules, and protein carbonyls reside in the cytosol, this decrease provided evidence that ERT exerted actions against intracellular, cytosolic, glucose-induced oxygen radicals. The related molecule MAN did not reduce protein carbonyls under high glucose conditions, which likely suggests that MAN did not reach the cytosol in sufficient levels.

Neither the exposure of the HUVEC cells to 5 mM ERT and 30 mM glucose (HGERT) nor to HG aloneaffected (p<0.1) malondialdehyde (MDA), an end product of lipid peroxidation. ERT was, however, able to inhibit radical-induced hemolysis (membrane protective effects) of erythrocytes exposed to hyperglycemic conditions within 24 h incubation. Thus, in HUVECs, under HGERT decreased protein carbonyls indicating that ERT may exert actions against intracellular, cytosolic, glucose-induced oxygen radicals.

One of the reactive oxygen species of which the production is likely to be increased during hyperglycemia is the superoxide radical (O₂•—), potentially due to an over-activation of the mitochondrial respiratory chain. Superoxide dismutases (SODs) constitute a family of enzymes that effectively reduces the amount of superoxide radicals by turning them into hydrogen peroxide and oxygen. SODs occur in the cytoplasma (copper, zinc SOD or SOD1) and in the mitochondrion (manganese SOD or SOD2). A third form (SOD3 or EC-SOD) is found extracellularly. A potential protective mechanism by which meso-1,2,3,4-tetrahydroxybutane improves and/or maintains cell function includes increasing the expression or activity of these SODs. Accordingly, the expression protein and activity levels of SOD2 (which is the inducible SOD) SOD1 and total SOD activity were studied.

It was previously discovered that ERT does not directly scavenge superoxide radicals in vitro. SOD parameters were therefore used to study whether ERT affects activity, expression, and protein levels of total SOD and SOD1 (cytoplasmic Cu—Zn SOD) and SOD2 [mitochondrial or manganese (Mn) SOD], enzymes degrading superoxide radicals to hydrogen peroxide and diatomic oxygen, thereby increasing NO biovailability and increasing vasorelaxation. The increased hydrogen peroxide in combination with reduced transition metal ions (e.g., Fe²⁺) was able to increase hydroxyl radicals, for ERT scavenging. The total SOD activity (U/mg protein), SOD1 activity, SOD2 activity, SOD2 expression (by RT-PCR), and SOD2 protein (by Western blot) were not affected by HGERT vs HG (p<0.1).

Meso-1,2,3,4-tetrahydroxybutane, during hyperglycemic but not during normoglycemic conditions, appeared to increase the total superoxide dismutase activity. Although the exposures and the SOD activity determination was carried out in five fold, this effect was not significant, mainly due to the high variation during the dual incubation with high glucose and ERT. Thus, it was found that ERT did not affect transcription or the protein levels of SOD2.

The addition of the peroxynitrite-generating compound, 3-morpholino sidnonimine-1 (SIN-1; SIN), increased the number of dead cells under NG (NG vs NGSIN) but not under HG conditions (HG vs HGSIN). In the cells pre-incubated with ERT, incubation with SIN-1 during NG (NGSIN vs NGSINERT) and HG (HGSIN vs HGSINERT) conditions decreased the amount of dead cells. The preincubation with MAN did not exhibit this protective property (NGSIN vs NGSINERT; HGSIN vs HGSINERT).

The addition of the advanced glycation end product CML during co-incubation with NG increased the amount of dead cells slightly (NG vs NGCML), which was not observed with HG (HG vs HGCML). The number of dead cells was not different when combined with ERT or MAN, as compared to the incubation with NG or HG+CML (NGCML vs NGCMLERT; HGCML vs HGCMLERT; NGCML vs NGCMLMAN; HGCML vs HGCMLMAN).

The following Table 1 summarizes the comparisons between the different cell combinations under the various treatments and preparations.

TABLE 1

| Treatment | Average | SEM | Comparison 1 | p value | Comparison 2 | p value |
|---|---|---|---|---|---|---|
| NG | 5.69 | 0.53 | | | | |
| NGERT | 10.08 | 2.08 | NG vs NGERT | 0.111 | | |
| NGMAN | 7.19 | 0.64 | NG vs NGMAN | 0.146 | NGERT vs | 0.255 |
| | | | | | NGMAN | |
| HG | 19.15 | 1.37 | NG vs HG | 0.001 | | |
| HGERT | 7.95 | 1.22 | HG vs HGERT | 0.004 | | |
| HGMAN | 13.87 | 3.61 | HG vs HGMAN | 0.243 | | |
| NGSIN | 12.29 | 2.55 | NG vs NGSIN | 0.065 | | |
| NGSINERT | 3.65 | 0.36 | NG vs | 0.299 | NGSIN vs | 0.029 |
| | | | NGSINERT | | NGSINERT | |
| NGSINMAN | 12.22 | 2.34 | NG vs | 0.035 | NGSIN vs | 0.985 |
| | | | NGSINMAN | | NGSINMAN | |
| HGSIN | 12.59 | 2.09 | HG vs HGSIN | 0.058 | | |
| HGSINERT | 6.73 | 0.69 | HGSIN vs | 0.056 | | |
| | | | HGSINERT | | | |
| HGSINMAN | 10.15 | 1.32 | HGSIN vs | 0.381 | HGSINERT vs | 0.084 |
| | | | HGSINMAN | | HGSINMAN | |
| NGCML | 8.12 | 0.92 | NG vs NGCML | 0.084 | | |
| NGCMLERT | 6.53 | 0.56 | NG vs | 0.337 | NGCML vs | 0.214 |
| | | | NGCMLERT | | NGCMLERT | |
| NGCMLMAN | 7.55 | 1.85 | NG vs | 0.389 | NGCML vs | 0.795 |
| | | | NGCMLMAN | | NGCMLMAN | |
| HGCML | 8.19 | 0.50 | HG vs HGCML | 0.002 | | |
| HGCMLERT | 5.75 | 1.53 | HGCML vs | 0.204 | | |
| | | | HGCMLERT | | | |
| HGCMLMAN | 9.42 | 0.37 | HGCML vs | 0.120 | HGCMLERT vs | 0.080 |
| | | | HGCMLMAN | | HGCMLMAN | |

T test, unpaired, 2-sided, equal variance
Bold values indicate a p value of <0.1

Eicosanoids

Another parameter evaluated in the targeted assays included the study of 23 eicosanoids in a comparison of HG versus HGERT conditions. The platelet vasoconstricting compound, TXB2, which is produced by platelets, and also endothelial cells was found to be increased from 0.76 (at HG) to 1.20 under HGERT condition.

Oxidative stress and high glucose incubations of endothelial cells have been shown to increase 12-HETE; and diabetic pigs with elevated blood glucose have increased 12-HETE. In human islet cells treated with 12(S)-HETE in vitro, there was decreased insulin secretion, increased cell death, and increased phosphorylated p38-MAPK (pp 38) protein activity. Addition of inflammatory cytokines increased pp 38. In monocytes, high glucose increased 12-HETE and monocyte adhesion to endothelial cells via monocytic production of integrins. In endothelial cells, 12-HETE induced endothelial cell integrin production in a PKC-dependent manner. Exposure of endothelial cells to 12-HETE was found to also reduce ability of the cells to produce the vasodilatory molecule prostacyclin. In the HUVEC cells, it is plausible that high glucose incubations induced inflammatory cytokines and oxidative stress, which triggered cell death and affected adhesion through signaling cascades involving 12-HETE. ERT partially reversed these effects, increasing cell survival and decreasing 12-HETE levels. Thus, it was found that the decrease in 12-HETE in pellets (HGERT vs HG) provides a mechanistic basis for survival, protection and anti-inflammatory properties of ERT, based on known properties of 12-HETE in endothelial cells exposed to HG or oxidative stress.

8-HETE is known to be produced in cultured endothelial cells. The decrease may be of biological importance since 8(S)-HETE is a natural agonist of PPARa and g. 8(S)-HETE also increases F-actin organization and epithelial wound closure in rat corneal epithelial cells, and could possibly have cytoskeletal effects in endothelial cells. Lastly, 8-HETE levels were reported to be increased in a keratinocyte cell line exposed to doxycycline-induced oxidative stress, and this led to inhibition of cell growth due to decreased DNA synthesis. The decrease in cell growth was mediated by p38 mitogen-activated protein kinase, but not ERK 1/2 or JNKISAP kinases. The signal transduction cascade in HUVEC cells exposed to 8-HETE could be directly investigated.

In studying the supernatants, 14,15-dihydroxy-5Z,8Z,11Z-eicosatrienoate (14,15-DiHETrE), produced from arachidonic acid via Cyp 2C and 2J to form epoxyeicosatrienoic acids (EETs), then converted to DiHETrE via sEH, decreased from 0.0085 to 0.0040 nM (p<0.05). This decrease in 14,15-DiHETrE is consistent with high glucose suppression of sEH, and should result in increased EETs, which would produce vasodilation. EETs were not, however, observed to be increased.

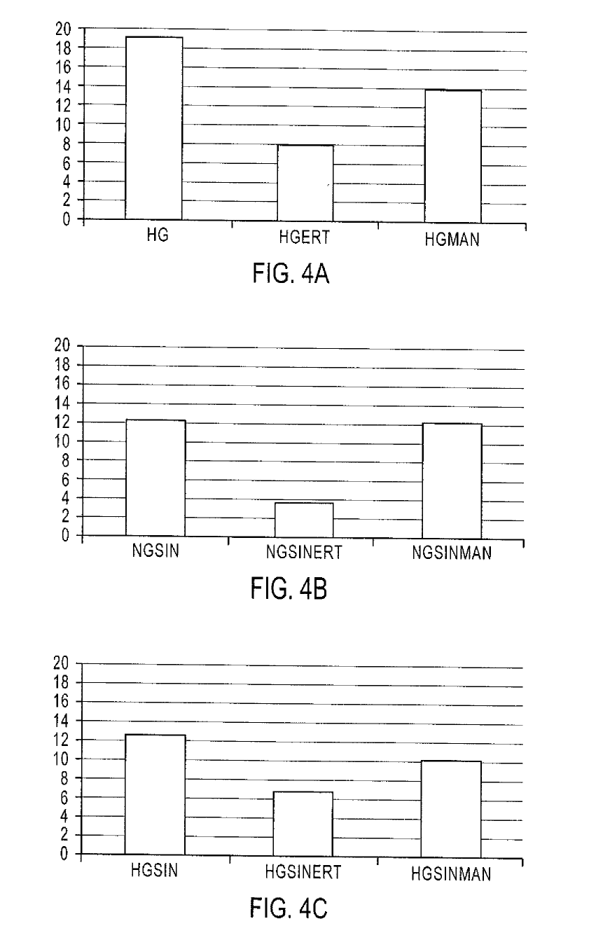

FIGS. 4A-4C provide a set of graphs illustrating the effects of meso-1,2,3,4-tetrahydroxybutane, decreasing the percentage of dead HUVEC cells. In FIG. 4A, HGERT is different from the leftmost bar (HG), p<0.05, T test, n=3 (no ERT added). In FIG. 4B, NGSINERT is different from the leftmost bar (NGSIN), p<0.05, T test, n=3 (no ERT added). In FIG. 4C, HGSINERT is different from the leftmost bar (HGSIN), p<0.05, T test, n=3 (no ERT added). In FIGS. 4A-4C, HG indicates 30 mM glucose for 24 hours. NG indicates 7 mM glucose for 24 hours. ERT indicates that ERT was added at 5 mM, 1 hour before stressors, and MAN indicates that MAN was added at 5 mM, 1 hour before stressors. SIN indicates 3-morpholino sidnonimine peroxynitrite generator, 24 hours.

### Example 2

Mouse intestine (entero-endocrine) STC-1 cells were incubated for 2 hours at 37° C., with fructose, glucose, sucrose, ERT, or 6-O-α-D-glucopyranosyl-D-fructose in duplicate, at 1%, 2%, 5%, and 10% w/v (final concentration). Appropriate blanks, positive controls, and standards were also included.

The lactate dehydrogenase (LDH) leakage from cells was analyzed in the supernatants as a marker for cytotoxic cell death. The 100% cell death corresponded to ca. 1200 U/L. The protein concentration in the 24 well plates (after the removal of the medium), was determined and used to normalize effects.

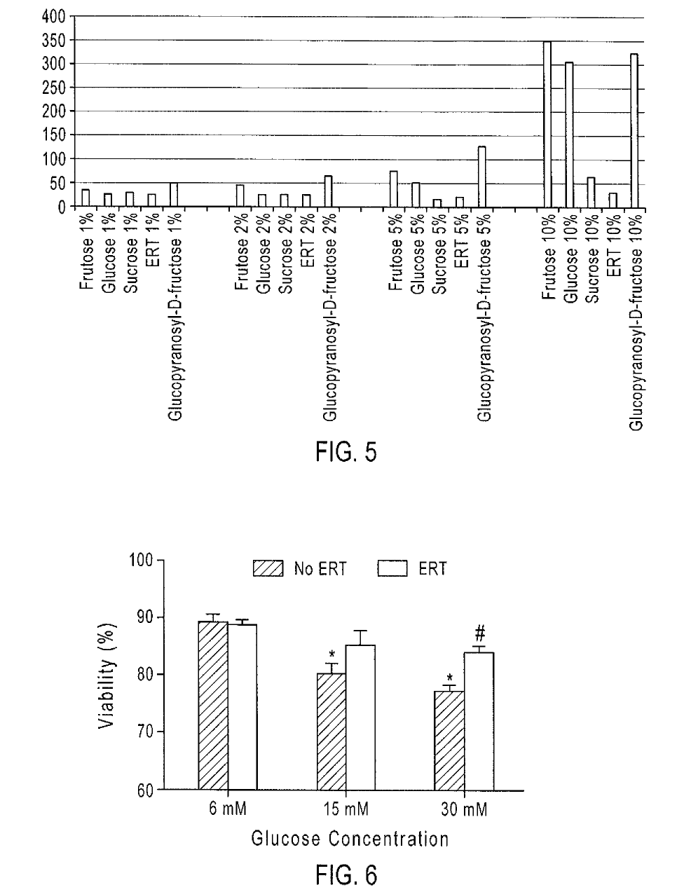

As indicated in FIG. 5, at a concentration high enough to induce significant stress (10%), cell death was higher with fructose, glucose, sucrose, and 6-O-α-D-glucopyranosyl-D-fructose than with ERT. The data indicated that these intestine cells could tolerate higher concentrations of ERT than glucose and other sugars tested, and that with ERT cell activity and functioning remained normal.

### Example 3

### Transcriptomics

Transcriptomics

The effect of high glucose with or without preincubation with meso-1,2,3,4,-tetrahydroxybutane on the transcriptome of the CRL 1730 HUVECs was investigated using DNA microarraytechnology, which is a powerful tool for comprehensive analysis of gene expression profiles of normal/disease states and the effects of drugs, nutraceuticals and food components on these states.

Accordingly, the last parameter evaluated included the number of transcript changes, Canonical pathways, super and sub categories and networks. Microarray transcriptomics were carried out on HUVEC cells exposed to varying glucose concentrations using the following exemplary method. In preparation of RNA isolation, the total RNA was isolated with Qiazol and purified with RNAeasy® Mini Kit (by Qiagen), using diethylprocarbonate (DEPC)-treated, RNase-free water. The purity of obtained RNA was tested spectrophotometrically (using Nanodrop) and determined suitable if 260/280 was >1.7. The integrity of obtained RNA was tested by lab-on-a-chip technology on an Agilent BioAnalyzer (Palo Alto, Calif.). The RNA had distinct 18S and 28S ribosomal RNA bands, which is a measure of intactness.

A sample of total RNA (10 μg) was reverse transcribed via Affymetrix protocols. The labeled cRNA was hybridized to Affymetrix Human Transcript U133 Plus 2.0 chips (P/N 520019; Lot No. 4096303). After the automated washing and staining, absolute values of expression were calculated from the scanned array using Affymetrix software.

Then the quality of chip hybridization and scanning was assessed using quality control (QC) functions within the Simpleaffy package (BioConductor). QC metrics assessed included average background; scale factor; number of transcripts called present; 3′ to 5′ ratios for β-actin and GAPDH; and values for spike-in control transcripts. The QC indicators were displayed in blue, indicating no RNA quality issues. As another measure of RNA quality, RNA degradation plots from the 12 samples used in microarray experiments showed similar intensities at 5′ and 3′ ends for different probes, with lower intensities at the more sensitive 5′ end, as expected.

After normalization of the data, a linear ANOVA model was applied in which the transcript expression˜glucose+treatment+glucose:treatment+day. The p-values were then calculated for the following comparisons: HGERT vs. HG (HGERT/HG); NGERT vs. NG (NGERT/NG); and HG vs. NG (HG/NG). The treatment had significant effects on transcript expression.

The results and data were collected. In comparing NG vs HG, under NG conditions, ERT was found to affect different and fewer transcripts and pathways. Comparing HG/NG vs HGERT/HG, surprisingly, it was found that ERT reverses many of the transciptomic changes observed in its absence, under HG conditions, in key pathways such as cell death. When studying cytosketal and focal adhesion transcriptomics, it was found that changes likely reflected adaptive survival and protection properties of the cell exposed to HGERT vs HG. It was found in the study of the polyinositide metabolism that diverse transcriptomic changes were related to survival and protection properties of the PI3K cascade and inositide-induced adaptive cytoskeletal changes, when comparing HGERT to HG.

Studies involving transcripts associated with cell survival and protectionor cell growth, showed that via diverse signalling pathways, under HG conditions, ERT promoted both cell survival and protection and cell death transcripts. Since ERT decreased cell death under HG conditions, it was concluded that post-translational events, not detected with transcriptomics, were involved in ERTs protective properties.

Studies involved in anti-inflammation, conducted under HG conditions, showed that ERT altered cytokine-related transcripts, with both pro-inflammatory and anti-inflammatory roles.

Transcriptomic evidence suggested that many transcripts were altered due to the transforming growth factor beta (TGFb) signalling pathway, in response to HGERT vs ERT.

Under HG conditions, it was found that ERT decreased flux through the citric acid cycle and electron transport complexes, which could decrease mitochondrial electron leakage leading to reactive oxygen species (ROS). However, classical markers of antioxidant or ROS scavenging were not detected with transcriptomics.

Tests surrounding purine and pyrimidine metabolism provided considerable transcriptomic evidence that ERT mediated effects via RNA polymerase to alter purine and pyrimidine metabolism, possibly to repair DNA and slow down transcription. Many changes to transcripts involved in pre-mRNA processing also indicated ERT affected transcription, which may be related to cell survival and protection properties. Under HG conditions, studies showed that ERT exerted cell survival and protection properties via modulating ER stress and unfolded protein response (UPR) (via sumoylation and de-ubiquitinization, for example) pathways related to protein degradation. Thus, overall, studies provided evidence that ERT is not a typical antioxidant. It does exert potent cell survival and protection properties under HG conditions and oxidant-stress conditions, with the latter based on targeted cell death assays.

The following Tables 2A-2F show the number of genes differentially expressed (at p<0.05) by HG vs NG and HGERT vs HG, for a variety of diseases, and includes data points on which genes were up or down regulated under high glucose stress conditions, and with which metabolic pathway these genes are associated with. The following Tables 2A-2F further demonstrate how ERT was able to reverse the up or down regulation of such genes under the same stress conditions, thereby reversing the stress related alterations of cell activity and functioning, and thereby reducing the risk of improper cell functioning or cell death, helping to maintain normal cell function and activity. In Tables 2A-2F, the ψ symbol indicates up regulation of a gene and Ω symbol indicates down regulation of a gene under the conditions HGERT/HG or HG/NG. A cell with no markings indicates that there was no change in the regulation of the gene under those conditions.

TABLE 2A
Diabetes Mellitus

| Pathway | Gene ID | Code | Name | HGERT/HG | HG/NG |
|---|---|---|---|---|---|
| Cysteine/gluathione | 10768 | AHCYL1 | Adenosylhomocysteinase-like 1 | Ψ 1.01 | |
| | 471 | ATIC | 5-aminoimidazole-4-carboxamide ribonucleotide | 0.99 Ω | Ψ 1.02 |
| | | | formyltransferase/IMP cyclohydrolase | | |
| | 2098 | ESD | Esterase D | 0.99 Ω | |
| Receptor activity | 152559 | PAQR3 | Progestin and adipoQ receptor family member III | 0.98 Ω | Ψ 1.03 |
| Focal adhesion | 1288 | COL4A6 | Collagen, type IV, alpha 6 | Ψ 1.03 | 0.99 Ω |
| PI3 kinase binding | 11151 | CORO1A | Coronin, actin binding protein, 1A | 0.94 Ω | |
| ATP binding and metabolism | 3984 | LIMK1 | LIM domain kinase 1 | Ψ 1.01 | |
| | 2011 | MARK2 | MAP/microtubule affinity-regulating kinase 2 | Ψ 1.03 | |
| | 6102 | RP2 | Retinitis pigmentosa 2 (X-linked recessive) | 0.98 Ω | |
| | 514 | ATP5E | ATP synthase, H+ transporting, mitochondrial F1 complex, | 0.99 Ω | Ψ 1.02 |
| | | | epsilon subunit | | |
| | 6196 | RPS6KA2 | Ribosomal protein S6 kinase, 90kDa, polypeptide 2 | Ψ 1.01 | 0.99 Ω |
| | 4067 | LYN | V-yes-1 Yamaguchi sarcoma viral related oncogene homolog | Ψ 1.02 | 0.99 Ω |
| | 3336 | HSPE1 | Heat shock 10kDa protein 1 (chaperonin 10) | 0.99 Ω | |
| | 53944 | CSNK1G1 | Casein kinase 1, gamma 1 | Ψ 1.02 | |
| | 27032 | ATP2C1 | ATPase, Ca++ transporting, type 2C, member 1 | 0.99 Ω | |
| | 23095 | KIF1B | Kinesin family member 1B | Ψ 1.01 | |
| | 23456 | ABCB10 | ATP-binding cassette, sub-family B (MDR/TAP), member 10 | 0.98 Ω | Ψ 1.02 |
| | 9525 | VPS4B | Vacuolar protein sorting 4 homolog B (S. cerevisiae) | 0.99 Ω | Ψ 1.02 |
| | 84896 | ATAD1 | ATPase family, AAA domain containing 1 | 0.99 Ω | |
| | 8803 | SUCLA2 | Succinate-CoA ligase, ADP-forming, beta subunit | 0.99 Ω | Ψ 1.02 |
| | 539 | ATP5O | ATP synthase, H+ transporting, mitochondrial F1 complex, | 1.00 Ω | |
| | | | O subunit | | |
| | 83858 | ATAD38 | ATPase family, AAA domain containing 3B | Ψ 1.01 | |
| | 57130 | ATP13A1 | ATPase type 13A1 | Ψ 1.02 | |
| | 84319 | C3orf26 | Chromosome 3 open reading frame 26 | 0.99 Ω | |
| | 79913 | ACTR5 | ARP5 actin-related protein 5 homolog (yeast) | Ψ 1.01 | |
| | 79072 | FASTKD3 | FAST kinase domains 3 | 0.99 Ω | |
| | 9748 | SLK | STE20-like kinase | 0.99 Ω | |
| | 79675 | FASTKD1 | FAST kinase domains 1 | 0.99 Ω | |
| | 6596 | HLTF | Helicase-like transcription factor | 0.99 Ω | Ψ 1.02 |
| | 86 | ACTL6A | Actin-like 6A | 0.98 Ω | |
| | 79877 | DCAKD | Dephospho-CoA kinase domain containing | Ψ 1.03 | |
| | 833 | CARS | Cysteinyl-tRNA synthetase | 1.00 Ω | Ψ 1.02 |
| | 79039 | DDX54 | DEAD (Asp-Glu-Ala-Asp) box polypeptide 54 | 0.99 Ω | |
| | 8491 | MAP4K3 | Mitogen-activated protein kinase kinase kinase kinase 3 | 0.98 Ω | |
| | 27347 | STK39 | Serine threonine kinase 39 | 0.98 Ω | |
| | 10730 | YME1L1 | YME1-like 1 (S. cerevisiae) | 1.00 Ω | |
| | 54802 | TRIT1 | TRNA isopentenyltransferase 1 | 0.98 Ω | |
| Reactive oxygen species | 10200 | MPHOSPH6 | M-phase phosphoprotein 6 | 0.99 Ω | |
| | 7052 | TGM2 | Transglutaminase 2 (C polypeptide, | Ψ 1.01 | |
| | | | protein-glutamine-gamma-glutamyltransferase) | | |
| | 54498 | SMOX | Spermine oxidase | 0.99 Ω | |
| | 3728 | JUP | Junction plakoglobin | Ψ 1.01 | |
| | 6938 | TCF12 | Transcription factor 12 | 0.99 Ω | Ψ 1.02 |
| | 1351 | COX8A | Cytochrome c oxidase subunit VIIIA (ubiquitous) | 0.99 Ω | |
| | 6654 | SOS1 | Son of sevenless homolog 1 (Drosophila) | Ψ 1.06 | 0.99 Ω |
| | 6392 | SDHD | Succinate dehydrogenase complex, subunit D, integral | 0.98 Ω | D:U |
| | | | membrane protein | | |
| Alpha-catenin binding | 3728 | JUP | Junction plakoglobin | Ψ 1.01 | |
| ILK signaling (integrin linked kinase) | 7431 | VIM | Vimentin | 1.00 Ω | |
| | 5829 | PXN | Paxillin | Ψ 1.01 | |
| | 87 | ACTN1 | Actinin, alpha 1 | Ψ 1.01 | |
| | 388 | RHOB | Ras homolog gene family, member B | Ψ 1.02 | 0.94 Ω |
| | 8440 | NCK2 | NCK adaptor protein 2 | Ψ 1.01 | 1.00 Ω |
| | 57381 | RHOJ | Ras homolog gene family, member J | Ψ1.02 | |
| | 54751 | FBLIM1 | Filamin binding LIM protein 1 | Ψ 1.02 | |
| | 6251 | RSU1 | Ras suppressor protein 1 | 0.99 Ω | |
| Sterol metabolism | 6646 | SOAT1 | Sterol O-acyltransferase 1 | 0.97 Ω | Ψ 1.02 |
| Apoptosis | 9500 | MAGED1 | Melanoma antigen family D, 1 | 1.00 Ω | |
| | 10081 | PDCD7 | Programmed cell death 7 | Ψ 1.02 | 0.99 Ω |
| | 83596 | Bcl2l12 | BCL2-like 12 (proline rich) | Ψ 1.02 | |
| Actin binding | 5339 | PLEC1 | Plectin | Ψ 1.03 | |
| | 11151 | CORO1A | Coronin, actin binding protein, 1A | 0.94 Ω | |
| Nitric oxide synthase activity | 29923 | HIG2 | Chromosome 7 open reading frame 68 | Ψ 1.01 | |
| | 2022 | ENG | Endoglin | Ψ 1.01 | 0.99 Ω |
| | 92 | ACVR2A | Activin A receptor, type IIA | 0.99 Ω | |
| Redox homeostasis | 51061 | TXNDC11 | Thioredoxin domain containing 11 | Ψ 1.01 | |
| | 889 | KRIT1 | KRIT1, ankyrin repeat containing | 0.99 Ω | |
| | 10954 | PDIA5 | Protein disulfide isomerase family A, member 5 | Ψ 1.01 | 0.99 Ω |
| | 10190 | TXNDC9 | Thioredoxin domain containing 9 | 0.99 Ω | Ψ 1.02 |

TABLE 2B
Alzhelmer's disease

| Paffmay | Gene ID | Code | Name | HGERT/HG | HG/NG |
|---|---|---|---|---|---|
| Apoptosis | 9500 | MAGED1 | Melanoma antigen family D, 1 | 1.00 Ω | |
| | 10081 | PDCD7 | Programmed cell death 7 | Ψ 1.02 | 0.99 Ω |
| | 83596 | Bcl2l12 | BCL2-like 12 (proline rich) | Ψ 1.02 | |
| Focal adhesion | 1288 | COL4A6 | Collagen, type IV, alpha 6 | Ψ 1.03 | 0.99 Ω |
| PI3 kinase binding | 11151 | CORO1A | Coronin actin binding protein, 1A | 0.94 Ω | |
| Cysteine/gluathione | 10768 | AHCYL1 | Adenosylhomocysteinase-like 1 | Ψ 1.01 | |
| | 471 | ATIC | 5-aminoimidazole-4-carboxamide ribonucleotide | 0.99 Ω | Ψ 1.02 |
| | | | formyltransferase/IMP cyclohydrolase | | |
| | 2098 | ESD | Esterase D | 0.99 Ω | |
| One Carbon metabolism | 10768 | AHCYL1 | Adenosylhomocysteinase-like 1 | Ψ 1.01 | |
| | 471 | ATIC | 5-aminiomidazole-4-carboxamide ribonucleotide | 0.99 Ω | 01.02 |
| | | | formyltransferase/IMP cyclohydrolase | | |
| | 2098 | ESD | Esterase D | 0.99 Ω | |
| ATP binding and metabolism | 3984 | LIMK1 | LIM domain kinase 1 | Ψ 1.01 | |
| | 2011 | MARK2 | MAP/microtubule affinity-regulating kinase 2 | Ψ 1.03 | |
| | 6102 | RP2 | Retinitis pigmentosa 2 (X-linked recessive) | 0.98 Ω | |
| | 514 | ATP5E | ATP synthase, H+ transporting, mitochondiral F1 complex, | 0.99 Ω | Ψ 1.02 |
| | | | epsilon subunit | | |
| | 6196 | RPS6KA2 | Ribosomal protein S6 kinase, 90kDa, polypeptide 2 | Ψ 1.01 | 0.99 Ω |
| | 4067 | LYN | V-yes-1 Yamaguchi sarcoma viral related oncogene homolog | Ψ 1.02 | 0.99 Ω |
| | 3336 | HSPE1 | Heat shock 10kDa protein 1 (chaperonin 10) | 0.99 Ω | |
| | 53944 | CSNK1G1 | Casein kinase 1, gamma 1 | Ψ 1.02 | |
| | 27032 | ATP2C1 | ATPase, Ca++ transporting, type 2C, member 1 | 0.99 Ω | |
| | 23095 | KIF18 | Kinesin family member 18 | Ψ 1.01 | |
| | 23456 | ABCB10 | ATP-binding cassette, sub-family B (MDR/TAP), member 10 | 0.98 Ω | Ψ 1.02 |
| | 9525 | VPS4B | Vacuolar protein sorting 4 homolog B (S. cerevisiae) | 0.99 Ω | Ψ 1.02 |
| | 84896 | ATAD1 | ATPase family, AAA domain containing 1 | 0.99 Ω | |
| | 8803 | SUCLA2 | Succinate-CoA ligase, ADP-forming, beta subunit | 0.99 Ω | Ψ 1.02 |
| | 539 | ATP5O | ATP synthase, H+ transporting, mitochondrial Fl complex, | 1.00 Ω | |
| | | | O subunit | | |
| | 83858 | ATAD3B | ATPase family, AAA domain containing 3B | Ψ 1.01 | |
| | 57130 | ATP13A1 | ATPase type 13A1 | Ψ 1.02 | |
| | 84319 | C3orf26 | Chromosome 3 open reading frame 26 | 0.99 Ω | |
| | 79913 | ACTR5 | ARP5 actin-related protein 5 homolog (yeast) | Ψ 1.01 | |
| | 79072 | FASTKD3 | FAST kinase domains 3 | 0.99 Ω | |
| | 9748 | SLK | STE20-like kinase | 0.99 Ω | |
| | 79675 | FASTKD1 | FAST kinase domains 1 | 0.99 Ω | |
| | 6596 | HLTF | Hellcase-like transcription factor | 0.99 Ω | Ψ 1.02 |
| | 86 | ACTL6A | Actin-like 6A | 0.98 Ω | |
| | 79877 | DCAKD | Dephospho-CoA kinase domain containing | Ψ 1.03 | |
| | 833 | CARS | Cysteinyl-tRNA synthetase | 1.00 Ω | Ψ 1.02 |
| | 79039 | DDX54 | DEAD (Asp-Glu-Ala-Asp) box polypeptide 54 | 0.99 Ω | |
| | 8491 | MAP4K3 | Mitogen-activated protein kinase kinase kinase kinase 3 | 0.98 Ω | |
| | 27347 | STK39 | Serine threonine kinase 39 | 0.98 Ω | |
| | 10730 | YME1L1 | YME1-like 1 (S. cerevisiae) | 1.00 Ω | |
| | 54802 | TRIT1 | TRNA Isopentenyltransferase 1 | 0.98 Ω | |
| Calcium/calmodulin | 3241 | HPCAL1 | Hippocalcin-like 1 | Ψ 1.01 | |
| | 22872 | SEC31A | SEC31 homolog A (S. cerevisiae) | Ψ 1.01 | |
| | 10519 | CIB1 | Calcium and integrin binding 1 (calmyrin) | 1.00 Ω | |
| | 29957 | SLC25A24 | Solute carrier family 25 (mitochondrial carrier; phosphate | 0.99 Ω | |
| | | | carrier), member 24 | | |
| | 10052 | GJC1 | Gap junction protein, gamma 1, 45kDa | Ψ 1.02 | |
| | 29078 | C6orf66 | NADH dehydrogenase (ubiquinone) 1 alpha subcomplex, | 0.99 Ω | |
| | | | assembly factor 4 | | |
| Caspase | 64105 | CENPK | Centromere protein K | 0.97 Ω | |
| | 3092 | HIP1 | Huntingtin interacting protein 1 | Ψ 1.05 | |
| | 2537 | IFI6 | Interferon, alpha-inducible protein 6 | Ψ 1.13 | |
| | 1611 | DAP | Death-associated protein | Ψ 1.01 | |
| | 9338 | TCEAL1 | Transcription elongation factor A (SII)-like 1 | 0.99 Ω | |
| | 4851 | NOTCH1 | Notch 1 | Ψ 1.02 | |
| | 6196 | RPS6KA2 | Ribosomal protein S6 kinase, 90kDa, polypeptide 2 | Ψ 1.01 | 0.99 Ω |
| | 4067 | LYN | V-yes-1 Yamaguchi sarcoma viral related oncogene homolog | Ψ 1.02 | 0.99 Ω |
| | 7431 | VIM | Vimentin | 1.00 Ω | |
| | 5829 | PXN | Paxillin | Ψ 1.01 | |
| | 10200 | MPHOSPH6 | M-phase phosphoprotein 6 | 0.99 Ω | |
| | 7052 | TGM2 | Transglutaminase 2 (C polypeptide, | Ψ 1.01 | |
| | | | protein-glutamine-gamma-glutamyltransferase) | | |
| | 3336 | HSPE1 | Heat shock 10kDa protein 1 (chaperonin 10) | 0.99 Ω | |
| | 2934 | GSN | Gelsolin | Ψ 1.03 | |
| Reactive oxygen Species | 10200 | MPHOSPH6 | M-phase phosphoprotein 6 | 0.99 Ω | |
| | 7052 | TGM2 | Transglutaminase 2 (C polypeptide, | Ψ 1.01 | |
| | | | protein-glutamine-gamma-glutamyltransferase) | | |
| | 54498 | SMOX | Spermine oxidase | 0.99 Ω | |
| | 3728 | JUP | Junction plakoglobin | Ψ 1.01 Ω | |
| | 6938 | TCF12 | Transcription factor 12 | 0.99 Ω | Ψ 1.02 |
| | 1351 | COX8A | Cytochrome c oxidase subunit VIIIA (ubiquitous) | 0.99 Ω | |
| | 6654 | SOS1 | Son of sevenless homolog 1 (Drosophila) | Ψ 1.06 | 0.99 Ω |
| | 6392 | SDHD | Succinate dehydrogenase complex, subunit D, integral | 0.98 Ω | D:U |
| | | | membrane protein | | |
| Cytoskeleton organization | 8440 | NCK2 | NCK adaptor protein 2 | Ψ 1.01 | 0.99 Ω |
| | 26499 | PLEK2 | Pleckstrin 2 | 0.96 Ω | |
| | 23622 | BACE1 | Beta-site APP-cleaving enzyme 1 | Ψ 1.02 | 0.99 Ω |
| | 9793 | CKAPS | Cytoskeleton associated protein 5 | Ψ 1.01 | |
| | 54820 | NDE1 | NudE nuclear distribution gene E homolog 1 (A. nidulans) | Ψ 1.01 | |
| | 10006 | AB [symbol] 1 | Ab [symbol] -interactor 1 | 0.99 Ω | |
| | 90410 | IFT20 | Intraflagellar transport 20 homolog (Chlamydomonas) | 1.00 Ω | |
| | 2801 | GOLGA2 | Golgin A2 | Ψ 1.04 | 0.99 Ω |
| | 79738 | BBS10 | Bardet-Biedl syndrome 10 | 0.98 Ω | |
| | 6867 | TACC1 | Transforming, acidic coiled-coil containing protein 1 | Ψ 1.01 | |
| | 11116 | FGFR1OP | FGFR1 oncogene partner | 0.99 Ω | Ψ 1.02 |
| | 6993 | DYNLT1 | Dynein, light chain, Tctex-type 1 | 0.99 Ω | Ψ 1.02 |
| Electron Transport Chain | 6391 | SDHC | Succinate dehydrogenase complex, subunit C, integral | 0.96 Ω | Ψ 1.02 |
| | | | membrane protein, 15kDa | | |
| | 55967 | NDUFA12 | NADH dehydrogenase (ubiquinone) 1 alpha subcomplex, 12 | 0.99 Ω | |
| | 79133 | C20orf7 | Chromosome 20 open reading frame 7 | 0.99 Ω | |
| | 7381 | UQCRB | Ubiquinol-cytochrome c reductase binding protein | 0.99 Ω | D:U |
| | 29796 | UCRC | Ubiquinol-cytochrome c reductase, complex III subunit X | 0.99 Ω | |
| Endothelial cell related | 4851 | NOTCH1 | Notch 1 | Ψ 1.02 | |
| | 889 | KRIT1 | KRIT1, ankyrin repeat containing | 0.99 | |
| | 25960 | GPR124 | G protein-coupled receptor 124 | Ψ 1.02 | 0.99 |
| | 2296 | FOXC1 | Forkhead box C1 | Ψ 1.01 | |
| | 29923 | HIG2 | Chromosome 7 open reading frame 68 | Ψ 1.01 | |
| | 2022 | ENG | Endoglin | Ψ 1.01 | 0.99 |
| | 3728 | JUP | Junction plakoglobin | Ψ 1.01 | |
| Fatty acid transport/metabolism | 3033 | HADH | Hydroxyacyl-CoA dehydrogenase | 0.99 | |
| | 1629 | DBT | Dihydrolipoamide branched chain transacylase E2 | Ψ 1.01 | |
| Ketone body metabolic process | 5019 | OXCT1 | 3-oxoacid CoA transferase 1 | 0.99 | |
| GLUT receptor | 2011 | MARK2 | MAP/microtubule affinity-regulating kinase 2 | Ψ 1.03 | |
| Protein transport | 25813 | SAMM50 | Sorting and assembly machinery component 50 homolog | 0.99 | |
| | | | (S. cerevisiae) | | |
| | 51025 | Magmas | Presequence translocase-associated motor 16 homolog | 0.98 | Ψ 1.02 |
| | | | (S. cerevisiae) | | |
| | 57511 | COG6 | Component of oligomeric golgi complex 6 | 0.99 | Ψ 1.02 |
| | 375035 | SFT2D2 | SFT2 domain containing 2 | 0.98 | |
| | 9559 | VPS26A | Vacuolar protein sorting 26 homolog A (S. pombe) | 0.99 | |
| | 91782 | CHMP7 | CHMP family, member 7 | 0.99 | |
| Glutamalergic signaling | 254263 | CNIH2 | Cornichon homolog 2 (Drosophila) | 0.99 | |
| | 7052 | TGM2 | Transglutaminase 2 (C polypeptide, | Ψ 1.01 | |
| | | | protein-glutamine-gamma-glutamyltransferase) | | |
| Hypoxia response | 23256 | SCFD1 | Sec1 family domain containing 1 | 0.99 | |
| Golgi post vesicle-mediated transport | 23256 | SCFD1 | Sec1 family domain containing 1 | 0.99 | |
| Heme biosynthesis | 51522 | TMEM14C | Transmembrane protein 14C | 0.99 | Ψ 1.02 |
| Histone acetylation | 10765 | [symbol] AR [symbol] D18 | Lysine (K)-specific demethylase 5B | Ψ 1.01 | 0.99 |
| | 23522 | MYST4 | MYST histone acetyltransferase (monocytic leukemia) 4 | Ψ 1.03 | |
| | 5396 | PRRX1 | Paired related homeobox 1 | Ψ 1.01 | |
| | 24149 | ZNF318 | Zinc finger protein 318 | Ψ 1.02 | |
| | 9767 | PHF16 | PHD finger protein 16 | 0.99 | |
| | 84193 | SETD3 | SET domain containing 3 | 0.99 | |
| | 64426 | SUDS3 | Suppressor of defective silencing 3 homolog (S. cerevisiae) | 0.99 | Ψ 1.02 |
| | 23135 | [symbol] M [symbol] D3 | Lysine (K)-specific demethylase 6B | Ψ 1.01 | |
| | 51562 | MBIP | MAP3KI2 binding inhibitory protein 1 | 0.98 | |
| | 56254 | RNF20 | Ring finger protein 20 | 0.99 | Ψ 1.02 |
| | 23326 | USP22 | Ubiquitin specific peptidase 22 | Ψ 1.01 | |
| Inositol metabolism | 65977 | PLEKHA3 | Pleckstrin homology domain containing, family A | 0.99 | |
| | | | (phosphoinositide binding specific) member 3 | | |
| | 4534 | MTM1 | Myotubularin 1 | 0.98 | |
| | 8724 | SNX3 | Sorting nexin 3 | 0.99 | |
| | 22908 | SACM1L | SAC1 suppressor of actin mutations 1-like (yeast) | 0.98 | Ψ 1.02 |
| | 57231 | SNX14 | Sorting nexin 14 | 0.99 | |
| | 84812 | PLCD4 | Phospholipase C, delta 4 | 0.99 | |
| | 285590 | SH3PXD2B | SH3 and PX domains 2B | Ψ 1.01 | |
| | 91404 | SESTD1 | SEC14 and spectrin domains 1 | Ψ 1.01 | |
| MAPKK activity | 2771 | GNAI2 | Guanine nucleotide binding protein (G protein), alpha | Ψ 1.02 | 0.99 |
| | | | inhibiting activity polypeptide 2 | | |
| | 57498 | KIDINS220 | Kinase D-interacting substrate, 220kDa | Ψ 1.01 | |
| Metal ion binding | 25939 | SAMHD1 | SAM domain and HD domain 1 | Ψ 1.07 | |
| | 9767 | PHF16 | PHD finger protein 16 | 0.99 | |
| | 10531 | PITRM1 | Pitrilysin metallopeptidase 1 | Ψ 1.01 | |
| | 5045 | FURIN | Furin (paired basic amino acid cleaving enzyme) | Ψ 1.02 | |
| | 51631 | LUC7L2 | LUC7-like 2 (S. cerevisiae) | 0.99 | |
| | 90850 | ZNF59B | Zinc finger protein 59B | 0.99 | |
| | 115825 | WDFY2 | WD repeat and FYVE domain containing 2 | Ψ 1.01 | |
| | 55139 | ANKZF1 | Ankyrin repeat and zinc finger domain containing 1 | 0.99 | Ψ 1.02 |
| | 54751 | FBLIM1 | Filamin binding LIM protein 1 | Ψ 1.02 | |
| | 23287 | AGTPBP1 | ATP/GTP binding protein 1 | 0.98 | |
| | 26521 | TIMM8B | Translocase of inner mitochondrial membrane 8 | 0.99 | |
| | | | homolog B (yeast) | | |
| | 7077 | TIMP2 | TIMP metallopeptidase inhibitor 2 | Ψ 1.01 | 0.99 |
| Mitotic cell cycle | 10200 | MPHOSPH6 | M-phase phosphoprotein 6 | 0.99 | |
| | 11190 | CEP250 | Centrosomal protein 250kDa | 0.99 | |
| | 9662 | CEP135 | Centrosomal protein 135kDa | Ψ 1.01 | |
| | 22994 | AZI1 | 5-azacytidine induced 1 | Ψ 1.02 | |
| | 11116 | FGFR1OP | FGFR1 oncogene partner | 0.99 | Ψ 1.02 |
| | 9039 | U8A3 | Ubiquitin-like modifier activating enzyme 8 | 0.99 | |
| NADH dehydrogenase | 55967 | NDUFA12 | NADH dehydrogenase (ubiquinone) 1 alpha subcomplex, 12 | 0.99 | |
| | 4697 | NDUFA4 | NADH dehydrogenase (ubiquinone) 1 alpha subcomplex, 4, | 0.99 | D:U |
| | | | 9kDa | | |
| Syntaxin binding | 63908 | NAP8 | N-ethylmaleimide-sensitive factor attachment protein, beta | 0.98 | |
| | 6814 | STXBP3 | Syntaxin binding protein 3 | 0.98 | |
| Nitric oxide synthase activity | 29923 | HIG2 | Chromosome 7 open reading frame 68 | Ψ 1.01 | |
| | 2022 | ENG | Endoglin | Ψ 1.01 | 0.99 |
| | 92 | ACVR2A | Activin A receptor, type IIA | 0.99 | |
| Peptidase activity | 26168 | SENP3 | SUMO1/sentrin/SMT3 specific peptidase 3 | Ψ 1.01 | |
| | 128488 | WFDC12 | WAP four-disulfide core domain 12 | 0.96 | Ψ 1.02 |
| | 4287 | ATXN3 | Ataxin 3 | 0.99 | |
| | 55593 | OTUD5 | OTU domain containing 5 | Ψ 1.01 | |
| Plasminogen activators | 57381 | RHOJ | Ras homolog gene family, member J | Ψ 1.02 | |
| | 6046 | BRD2 | Bromodomain containing 2 | Ψ 1.02 | 0.99 |
| Redox homeostasis | 51061 | TXNDC11 | Thioredoxin domain containing 11 | Ψ 1.01 | |
| | 889 | KRIT1 | KRIT1, ankyrin repeat containing | 0.99 | |
| | 10954 | PDIA5 | Protein disulfide isomerase family A, member 5 | Ψ 1.01 | 0.99 |
| | 10190 | TXDC9 | Thioredoxin domain containing 9 | 0.99 | Ψ 1.02 |
| SMAD binding | 163326 | EID2 | EP300 interacting inhibitor of differentiation 2 | 0.99 | |
| Transmembrane transport | 84102 | SLC41A2 | Solute carrier family 41, member 2 | 0.99 | |
| | 55676 | SLC30A6 | Solute carrier family 30 (zinc transporter), member 6 | 0.99 | |
| | 119559 | SFXN4 | Sideroflexin 4 | 0.98 | |
| | 23480 | SEC61G | Sec61 gamma subunit | 0.99 | |
| | 154091 | SLC2A12 | Solute carrier family 2 (facilitated glucose transporter), | Ψ 1.03 | |
| | | | member 12 | | |
| | 64747 | MFSOL | Major facilitator superfamily domain containing 1 | 0.99 | |
| | 203427 | SLC25A43 | Solute carrier family 25, member 43 | 0.98 | Ψ 1.02 |
| | 84641 | HIATL1 | Hippocampus abundant transcript-like 1 | 0.99 | |
| Ubiquinol-cytochrome-c | 7381 | UQCRB | Ubiquinol-cytochrome c reductase binding protein | 0.99 | Ψ 1.02 |
| reductase activity | 29796 | UCRC | Ubiquinol-cytochrome c reductase, complex III subunit X | 0.99 | |

[symbol]

indicates data missing or illegible when filed

TABLE 2C
Prostate cancer

| Pathway | Gene ID | Code | Name | HGERT/HG | HG/NG |
|---|---|---|---|---|---|
| Actin binding | 5339 | PLEC1 | Plectin | Ψ 1.03 | |
| | 11151 | CORO1A | Coronin, actin binding protein, 1A | 0.94 Ω | |
| ILK signaling (integrin linked kinase) | 7431 | VIM | Vimentin | 1.00 Ω | |
| | 5829 | PXN | Paxillin | Ψ 1.01 | |
| | 87 | ACTN1 | Actinin, alpha 1 | Ψ 1.01 | |
| | 388 | RHOB | Ras homolog gene family, member B | Ψ 1.02 | 0.94 Ω |
| | 8440 | NCK2 | NCK adaptor protein 2 | Ψ 1.01 | 1.00 Ω |
| | 57381 | RHOJ | Ras homolog gene family, member J | Ψ 1.02 | |
| | 54751 | FBUM1 | F [symbol] amin binding UM protein 1 | Ψ 1.02 | |
| | 6251 | RSU1 | Ras suppressor protein 1 | 0.99 Ω | |
| Endothelial cell-cell adhesion | 3728 | JUP | Junction plakoglobin | Ψ 1.01 | |
| Reactive oxygen species | 10200 | MPHOSPH6 | M-phase phosphoprotein 6 | 0.99 Ω | |
| | 7052 | TGM2 | Transglutaminase 2 (C polypeptide, | Ψ 1.01 | |
| | | | protein-glutamine-gamma-glutamyltransferase) | | |
| | 54498 | SMOX | Spermine oxidase | 0.99 Ω | |
| | 3728 | JUP | Junction plakaglobin | Ψ 1.01 | |
| | 6938 | TCF12 | Transcription factor 12 | 0.99 Ω | Ψ 1.02 |
| | 1351 | COX8A | Cytochrome c oxidase subunit VIIIA (ubiquitous) | 0.99 Ω | |
| | 6654 | SOS1 | Son of sevenless homolog 1 (Drosophila) | Ψ 1.06 | 0.94 Ω |
| | 6392 | SDHD | Succinate dehydrogenase complex, subunit D, | 0.98 Ω | Ψ 1.02 |
| | | | integral membrane protein | | |
| Carbohydrate binding | 8702 | B4GALT4 | UDP-Gal:betaGlcNAc beta 1,4-galactosyltransferase, | 0.98 Ω | |
| | | | polypeptide 4 | | |
| | 282969 | C10orf125 | Chromosome 10 open reading frame 125 | 0.99 Ω | |
| | 2137 | EXTL3 | Exostoses (multiple)-like 3 | Ψ 1.02 | |
| | 7841 | GCS2 | Mannosyl-oligosaccharide glucosidase | Ψ 1.02 | |
| Receptor activity | 157559 | PAQR3 | Progestin and adipoQ receptor family member III | 0.98 Ω | Ψ 1.02 |
| Steroid hormone receptor activity | 10424 | PGRMC2 | Progesterone receptor membrane component 2 | 0.98 Ω | |
| Sterol metabolism | 6646 | SOAT1 | Sterol O-acyltransferase 1 | 0.97 Ω | Ψ 1.02 |
| Tyrosine phosphatase activity | 51495 | PTPLAD1 | Protein tyrosine phosphatase-like A domain containing 1 | Ψ 1.01 | |
| | 54935 | DUSP23 | Dual specificity phosphatase 23 | 0.99 Ω | |
| 8MP signaling pathway | 654 | BMP6 | Bone morphogenetic protein 6 | Ψ 1.01 | |
| | 7321 | UBE2D1 | Ubiquitin-conjugating enzyme E2D1 (U8C4/5 homolog, yeast) | 0.99 Ω | |
| Calcium/calmodulin | 3241 | HPCAL1 | Hippocalcin-like 1 | Ψ 1.01 | |
| | 22872 | SEC31A | SEC31 homolog A (S. cerevisiae) | Ψ 1.01 | |
| | 10519 | CIB1 | Calcium and integrin binding 1 (calmyrin) | Ψ 1.00 | |
| | 29957 | SLC25A24 | Solute carrier family 25 (mitochondrial carrier; phosphate | 0.99 Ω | |
| | | | carrier), member 24 | | |
| | 10052 | GJC1 | Gap junction protein, gamma 1, 45kDa | Ψ 1.02 | |
| | 29078 | C6orf66 | NADH dehydrogenase (ubiquinone) 1 alpha subcomplex, | 0.99 Ω | |
| | | | assembly factor 4 | | |
| DNA binding | 1112 | FOXN3 | Forkhead box N3 | Ψ 1.01 | 0.94 Ω |
| | 103 | ADAR | Adenosine deaminase, RNA-specific | Ψ 1.01 | |
| | 5440 | POLR2K | Polymerase (RNA) II (DNA directed) polypeptide K, 7.0kDa | 0.98 Ω | |
| | 10308 | ZNF267 | Zinc finger protein 267 | 0.98 Ω | Ψ 1.02 |
| | 284459 | HKR1 | HKR1, GU-Kruppel zinc finger family member | 0.98 Ω | |
| | 55769 | ZNF83 | Zinc finger protein 83 | 0.98 Ω | |
| | 3344 | FOXN2 | Forkhead box N2 | 0.98 Ω | |
| | 80110 | ZNF614 | Zinc finger protein 614 | 0.98 Ω | |
| | 84324 | SARNP | SAP domain containing ribonucleoprotein | 0.98 Ω | |
| | 92399 | MRRF | Mitochondrial ribosome recycling factor | 0.98 Ω | |
| | 147808 | ZNFT84 | Zinc finger protein 784 | 0.99 Ω | |
| | 7726 | TRIM26 | Tripartite motif-containing 26 | 0.99 Ω | |
| | 65056 | GPBP1 | GC-rich promoter binding protein 1 | 0.99 Ω | Ψ 1.02 |
| | 57491 | pdcd6 | Aryl-hydrocarbon receptor repressor | 0.99 Ω | |
| | 22980 | TCF25 | Transcription factor 25 (basic helix-loop-helix) | Ψ 1.01 | |
| | 64412 | GZF1 | GDNF-inducible zinc finger protein 1 | Ψ 1.01 | |
| | 7775 | ZNF232 | Zinc finger protein 232 | Ψ 1.01 | |
| | 84327 | ZBED3 | Zinc finger, BED-type containing 3 | Ψ 1.01 | 0.94 Ω |
| | 9878 | TOX4 | TOX high mobility group box family member 4 | Ψ 1.01 | |
| | 6659 | SOX4 | SRY (sex determining region Y)-box 4 | Ψ 1.01 | 0.94 Ω |
| | 27086 | FOXP1 | Forkhead box P1 | Ψ 1.01 | |
| | 56987 | BBX | Bobby sox homolog (Drosophilia) | Ψ 1.02 | |
| | 6928 | HNF1B | HNF1 homeobox B | Ψ 1.03 | |
| | 54811 | ZNF562 | Zinc finger protein 562 | Ψ 1.03 | 0.94 Ω |
| | 22827 | PUF60 | Poly-U binding splicing factor 60KDa | Ψ 1.01 | |
| | 1629 | DBT | Dihydrolipoamide branched chain transacylase E2 | Ψ 1.01 | |
| | 84897 | T8RG1 | Transforming growth factor beta regulator 1 | Ψ 1.02 | 0.94 Ω |
| | 29855 | UBN1 | Ubinuclein 1 | Ψ 1.02 | |
| | 22944 | KIN | KIN, antigenic determinant of recA protein homolog (mouse) | 0.99 Ω | |
| | 27342 | RABGEF1 | RAB guanine nucleotide exchange factor (GEF) 1 | 0.99 Ω | |
| | 10522 | DEAF1 | Deformed epidermal autoregulatory factor 1 (Drosophila) | Ψ 1.02 | |
| | 10608 | MXD4 | MAX dimerization protein 4 | Ψ 1.02 | 0.94 Ω |
| | 10621 | POLR3F | Polymerase (RNA) III (DNA directed) polypeptide F, 39 kDa | 0.98 Ω | |
| | 6119 | RPA3 | Replication protein A3, 14kDa | 0.99 Ω | |
| | 5356 | PRRX1 | Paired related homeobox 1 | Ψ 1.01 | |
| | 22926 | ATF6 | Activating transcription factor 6 | Ψ 1.01 | |
| | 2296 | FOXC1 | Forkhead box C1 | Ψ 1.01 | |
| | 5428 | POLG | Polymerase (DNA directed), gamma | Ψ 1.01 | |
| | 5424 | POLO1 | Polymerase (DNA directed), delta 1, catalytic subunit 125kDa | Ψ 1.02 | |
| | 5093 | PCBP1 | Poly(rC) binding protein 1 | Ψ 1.01 | |
| | 6938 | TCF12 | Transcription factor 12 | 0.99 Ω | Ψ 1.02 |
| | 6654 | SOS1 | Son of sevenless homolog 1 (Drosophila) | Ψ 1.06 | 0.94 Ω |
| | 26959 | HBP1 | HMG-box transcription factor 1 | 0.99 Ω | |
| | 26994 | RNF11 | Ring finger protein 11 | 0.99 Ω | |
| | 7270 | TTF1 | Transcription termination factor, RNA polyrnerase 1 | Ψ 1.01 | 0.94 Ω |
| | 51588 | PIAS4 | Protein inhibitor of activated STAT 4 | Ψ 1.03 | |
| | 23429 | RYBP | RING1 and YY1 binding protein | Ψ 1.01 | U:D |
| | 26994 | RNF11 | Ring finger protein 11 | 0.99 Ω | |
| | 7270 | TTF1 | Transcription termination factor, RNA polymerase 1 | Ψ 1.01 | 0.94 Ω |
| | 51588 | PIAS4 | Protein inhibitor of activated STAT, 4 | Ψ 1.03 | |
| | 23429 | RYBP | RING1 and YY1 binding protein | Ψ 1.01 | 0.94 Ω |
| Zinc ion transport | 55676 | SLC30A6 | Solute carrer family 30 (zinc transporter), member 6 | 0.99 Ω | |
| | 10463 | SLC30A9 | Solute carrer family 30 (zinc transporter), member 9 | 0.99 Ω | |
| | 91252 | SLC39A13 | Solute carrer family 39 (zinc transporter), member 13 | Ψ 1.01 | |

[symbol]

indicates data missing or illegible when filed

TABLE 2D
Breast cancer

| Pathway | Gene ID | Code | Name | HGERT/HG | HG/NG |
|---|---|---|---|---|---|
| Actin binding | 5339 | PLEC1 | Plectin | Ψ 1.03 | |
| | 11151 | CORO1A | Coronin, actin binding protein, 1A | 0.94 Ω | |
| Heat shock protein | 55735 | DNAJC11 | DnaJ (Hsp40) homolog, subfamily C, member 11 | Ψ 1.01 | |
| | 22826 | DNAJC8 | DnaJ (Hsp40) homolog, subfamily C, member 8 | 0.99 Ω | |
| Receptor activity | 152559 | PAQR3 | Progestin and adipoQ receptor family member III | 0.98 Ω | Ψ 1.03 |
| Steroid hormone receptor activity | 10424 | PGRMC2 | Progesterone receptor membrane component 2 | 0.98 Ω | |
| Tyrosine phosphatase activity | 51495 | PTPLAD1 | Protein tyrosine phosphatase-like A domain containing 1 | Ψ 1.01 | |
| | 54935 | DUSP23 | Dual specificity phosphatase 23 | 0.99 Ω | |
| Focal adhesion | 1288 | COL4A6 | Collagen, type IV, alpha 6 | Ψ 1.03 | 0.98 Ω |
| Insulin binding | 7431 | VIM | Vimentin | 1.00 Ω | |
| | 5829 | PXN | Paxillin | Ψ 1.01 | |
| | 87 | ACTN1 | Actinin, alpha 1 | Ψ 1.01 | |
| | 380 | RHOB | Ras homolog gene family, member B | Ψ 1.02 | 0.98 Ω |
| | 8440 | NCK2 | NCK adaptor protein 2 | Ψ 1.01 | 0.98 Ω |
| | 57381 | RHOJ | Ras homolog gene family, member J | Ψ 1.02 | |
| | 11180 | WDRG | WD repeat domain 6 | Ψ 1.02 | 0.98 Ω |
| | 5295 | PIK3R1 | Phosphoinositide-3 kinase, regulatory subunit 1 (alpha) | Ψ 1.03 | |
| | 54751 | FBLM1 | Filamin binding LIM protein 1 | Ψ 1.02 | |
| | 6251 | RSU1 | Ras suppressor protein 1 | 0.99 Ω | |
| PI3 kinase binding | 11151 | CORO1A | Coronin, actin binding protein, 1A | 0.94 Ω | |
| ATP metabolism | 3984 | LIMK1 | LIM domain kinase 1 | Ψ 1.01 | |
| | 2011 | MARK2 | MAP/microtubule affinity-regulating kinase 2 | Ψ 1.03 | |
| | 5102 | RP2 | Retinitis pigmentosa 2 (X-linked recessive) | 0.98 Ω | |
| | 514 | ATP5E | ATP synthase, H+ transporting, mitochondrial F1 complex, | 0.99 Ω | Ψ 1.03 |
| | | | epsilon subunit | | |
| | 6196 | RPS6KA2 | Ribosomal protein S6 kinase, 90kDa, polypeptide 2 | Ψ 1.01 | 0.98 Ω |
| | 4067 | LYN | V-yes-1 Yamaguchi sarcoma viral related oncogene homolog | Ψ 1.02 | 0.98 Ω |
| | 3336 | HSPE1 | Heat shock 10kDa protein 1 (chaperonin 10) | 0.99 Ω | |
| | 53944 | CSNK1G1 | Casein kinase 1, gamma 1 | Ψ 1.02 | |
| | 27032 | ATP2C1 | ATPase, Ca++ transporting, type 2C, member 1 | 0.99 Ω | |
| | 23095 | KF1B | Kinesin family member 1B | Ψ 1.01 | |
| | 23456 | ABCB10 | ATP-binding cassette, sub-family B (MDR/TAP), member 10 | 0.98 Ω | Ψ 1.03 |
| | 9525 | VPS4B | Vacuolar protein sorting 4 homolog B (S. cerevisiae) | 0.99 Ω | Ψ 1.03 |
| | 84896 | ATAD1 | ATPase family, AAA domain containing 1 | 0.99 Ω | |
| | 8803 | SUCLA2 | Succinate-CoA ligase, ADP-forming, beta subunit | 0.99 Ω | Ψ 1.03 |
| | 539 | ATP5O | ATP synthase, H+ transporting, mitochondrial F1 | Ψ 1.00 | |
| | | | complex, O subunite | | |
| | 83858 | ATAD38 | ATPase family, AAA domain containing 38 | Ψ 1.01 | |
| | 57130 | ATP13A1 | ATPase type 13A1 | Ψ 1.02 | |
| | 84319 | C3orf26 | Chromosome 3 open reading frame 26 | 0.99 Ω | |
| | 79913 | ACTR5 | ARP5 actin-related protein 5 homolog (yeast) | Ψ 1.01 | |
| | 79072 | FASTKD3 | FAST kinase domains 1 | 0.99 Ω | |
| | 9748 | SLK | STE20-like kinase | 0.99 Ω | |
| | 79675 | FASTDK1 | FAST kinase domains 1 | 0.99 Ω | |
| | 6596 | HLTF | Helicase-like transcription factor | 0.99 Ω | Ψ 1.03 |
| | 86 | ACTL6A | Actin like 6A | 0.98 Ω | |
| | 79877 | DCAKO | Dephospho-CoA kinase domain containing | Ψ 1.03 | |
| | 833 | CARS | Cysteinyl-tRNA synthetase | 1.00 Ω | 0.98 Ω |
| | 79039 | DDX54 | DEAD (Asp-Glu-Ala-Asp) box, polypeptide 54 | 0.99 Ω | |
| | 8491 | MAP4K3 | Mitogen-activated protein kinase kinase kinase kinase 3 | 0.98 Ω | |
| | 27347 | STK39 | Serine threonine kinase 39 | 0.98 Ω | |
| | 10730 | YME1L1 | YME1-like 1 (S. cerevisiae) | 1.00 Ω | |
| | 54802 | TRIT1 | TRNA isopentenyltransferase 1 | 0.98 Ω | |
| Cysteine/gluathione | 10768 | AHCYL1 | Adenosylhomocysteinase-like 1 | Ψ 1.01 | |
| | 471 | ATIC | 5-aminoimidazole-4-carboxamide ribonucleotide | 0.99 Ω | Ψ 1.03 |
| | | | formyltransferase/IMP cyclohydroiase | | |
| | 2098 | ESD | Esterase D | 0.99 Ω | |
| One carbon metabolism | 10768 | AHCYL1 | Adenosylhomocysteinase-like 1 | Ψ 1.01 | |
| | 471 | ATIC | 5-aminoimdazole-4-carboxamide ribonucleotide | 0.99 Ω | Ψ 1.03 |
| | | | formyltransferase/IMP cyclohydroiase | | |
| | 2098 | ESD | Esterase D | 0.99 Ω | |
| Alpha-catenin binding | 3728 | JUP | Junction plakoglobin | Ψ 1.01 | |
| Deoxycytidine metabolism | 1633 | DCK | Deoxycytidine kinase | 0.98 Ω | Ψ 1.01 |
| Hypoxia response | 23256 | ACTN1 | Sec1 family domain containing 1 | 0.99 Ω | |
| ILK signaling (integrin linked kinase) | 7431 | VIM | Vimentin | 1.00 Ω | |
| | 5829 | PXN | Paxillin | Ψ 1.01 | |
| | 87 | ACTN1 | Actinin, alpha 1 | Ψ 1.01 | |
| | 388 | RHOB | Ras homolog gene family, member B | Ψ 1.02 | 0.98 Ω |
| | 8440 | NCK2 | NCK adaptor protein 2 | Ψ 1.01 | 0.98 Ω |
| | 57381 | RHOJ | Ras homolog gene family, member J | Ψ 1.02 | |
| | 54751 | FBLIM1 | Flamin binding LIM protein 1 | Ψ 1.02 | |
| | 6251 | RSU1 | Ras suppressor protein 1 | 0.99 Ω | |
| Mitotic cell cycle | 10200 | MPHOSPH6 | M-phase phosphoprotein G | 0.99 Ω | |
| | 11190 | CEP250 | Centrosomal protein 250kDa | 0.99 Ω | |
| | 9662 | CEP135 | Centrosomal protein 135kDa | Ψ 1.01 | |
| | 22994 | AZI1 | S-azacytidine induced 1 | Ψ 1.02 | |
| | 11115 | FGFR1OP | FGFR1 oncogene partner | 0.99 Ω | Ψ 1.03 |
| | 9039 | UBA3 | Ubiquitin-like modifier activating enzyme 3 | 0.99 Ω | |
| Osteoclast differentiation | 28962 | OSTM1 | Osteopetrosis associated transmembrane protein 1 | 0.99 Ω | |
| Peptidase activity | 26168 | SENP3 | SUMO1/sentrin/SMT3 sepcific peptidase 3 | Ψ 1.01 | |
| | 128488 | WFDC12 | WAP four-disulfide core domain 12 | 0.96 Ω | Ψ 1.03 |
| | 4287 | ATXN3 | Ataxin 3 | 0.99 Ω | |
| | 55593 | OTUD5 | OTU domain containing 5 | Ψ 1.01 | |
| Reactive oxygen species | 10200 | MPHOSPH6 | M-phase phosphoprotein 6 | 0.99 Ω | |
| | 7052 | TGM2 | Transglutaminase 2 (C polypeptide, | Ψ 1.01 | |
| | | | protein-glutamine-gamma-glutamyltransferase) | | |
| | 54498 | SMOX | Spermine oxidase | 0.99 Ω | |
| | 3728 | JUP | Junction plankoglobin | Ψ 1.01 | |
| | 6938 | TCF12 | Transcription factor 12 | 0.99 Ω | Ψ 1.03 |
| | 1351 | COX8A | Cytochrome c oxidase subunit VIIIA (ubiquitous) | 0.99 Ω | |
| | 6654 | SOS1 | Son of sevenless homolog 1 (Drosophila) | Ψ 1.06 | 0.99 Ω |
| | 6392 | SDHD | Succinate dehydrogenase complex, subunit D, integral | 0.98 Ω | Ψ 1.01 |
| | | | membrane protein | | |
| SMAD binding | 163126 | EID2 | EP300 interacting inhibitor of differentiation 2 | 0.99 Ω | |
| Sulfotransferase acfivity | 9957 | HS3ST1 | Heparan sulfate (glucosamine) 3-O-sulfotransferase 1 | Ψ 1.03 | |
| Plasminogen activators | 57381 | RHOJ | Ras homolog gene family, member J | Ψ 1.02 | |
| | 6046 | BAD2 | Bromodomain containing 2 | Ψ 1.02 | 0.99 Ω |

TABLE 2E
Atherosclerosis

| Pathway | Gene ID | Code | Name | HGERT/HG | HG/NG |
|---|---|---|---|---|---|
| Steroid hormone receptor activity | 10424 | PGRMC2 | Progesterone receptor membrane component 2 | 0.98 Ω | |
| Cysteine/gluathione | 10768 | AHCYL1 | Adenosylhomocysteinase-like 1 | Ψ 1.01 | |
| | 471 | ATIC | 5-aminoimidazole-4-carboxamide ribonucleotide | 0.99 Ω | Ψ 1.01 |
| | | | formyltransferase/IMP cyclohydrolase | | |
| | 2098 | ESD | Esterase D | 0.99 Ω | |
| Osteoclast differentiation | 28962 | OSTM1 | Osteopetrosis associated transmembrane protein 1 | 0.99 Ω | |
| Peptidase activity | 26168 | SENP3 | SUMO1/sentrin/SMT3 specific peptidase 3 | Ψ 1.01 | |
| | 128488 | WFDC12 | WAP four-disulfide core domain 12 | 0.96 Ω | Ψ 1.03 |
| | 4287 | ATXN3 | Ataxin 3 | 0.99 Ω | |
| | 55593 | OTUD5 | OTU domain containing 5 | Ψ 1.01 | |
| Reactive oxygen species | 10200 | MPHOSPH6 | M-phase phosphoprotein 6 | 0.99 Ω | |
| | 7052 | TGM2 | Transglutaminase 2 (C polypeptide, | Ψ 1.01 | |
| | | | protein-glutamine-gamma-glutamyltransferase) | | |
| | 54498 | SMOX | Spermine oxidase | 0.99 Ω | |
| | 3728 | JUP | Junction plakoglobin | Ψ 1.01 | |
| | 6938 | TCF12 | Transcription factor 12 | 0.99 Ω | Ψ 1.01 |
| | 1351 | COX8A | Cytochrome c oxidase subunit VIIIA (ubiquitous) | 0.99 Ω | |
| | 6654 | SOS1 | Son of sevenless homolog 1 (Drosophila) | Ψ 1.06 | 0.98 Ω |
| | 6392 | SDHD | Succinate dehydrogenase complex, subunit D, | 0.98 Ω | Ψ 1.03 |
| | | | integral membrane protein | | |
| Plasminogen activators | 57381 | RHOJ | Ras homolog gene family, member J | Ψ 1.02 | |
| | 6046 | BRD2 | Bromodomain containing 2 | Ψ 1.02 | 0.99 Ω |
| Endothelial cell-cell adhesion | 3728 | JUP | Junction plakoglobin | Ψ 1.01 | |
| ATP binding and metabolism | 3984 | LIMK1 | LIM domain kinase 1 | Ψ 1.01 | |
| | 2011 | MARK2 | MAP/microtubule affinity-regulating kinase 2 | Ψ 1.03 | |
| | 6102 | RP2 | Retinitis pigmentosa 2 (X-linked recessive) | 0.99 Ω | Ψ 1.01 |
| | 514 | ATP5E | ATP synthase, H+ transporting, mitochondrial F1 | 0.98 Ω | Ψ 1.01 |
| | | | complex, epsilon subunit | | |
| | 6196 | RPS6KA2 | Ribosomal protein S6 kinase, 90kDa, polypeptide 2 | Ψ 1.01 | U:D |
| | 4067 | LVN | V-yes-1 Yamaguchi sarcoma viral related oncogene | Ψ 1.02 | 0.99 Ω |
| | | | homolog | | |
| | 3336 | HSPE1 | Heat shock 10kDa protein 1 (chaperonin 10) | 0.99 Ω | |
| | 53944 | CSNK1G1 | Casein kinase 1, gamma 1 | Ψ 1.02 | |
| | 27032 | ATP2C1 | ATPase, Ca++ transporting, type 2C, member 1 | 0.99 Ω | |
| | 23095 | KIF1B | Kinesin family member 1B | Ψ 1.01 | |
| | 23456 | ABCB10 | ATP-binding cassette, sub-family B (MDR/TAP), | 0.98 Ω | Ψ 1.01 |
| | | | member 10 | | |
| | 9525 | VPS4B | Vacuolar protein sorting 4 homolog B (S. cerevisiae) | 0.99 Ω | Ψ 1.01 |
| | 84896 | ATAD1 | ATPase family, AAA domain containing 1 | 0.99 Ω | |
| | 8803 | SUCLA2 | Succinate-CoA ligase, ADP-forming, beta subunit | 0.99 Ω | Ψ 1.03 |
| | 539 | ATP5O | ATP synthase, H+ transporting, mitochondrial Fl | 1.00 Ω | |
| | | | complex, O subunit | | |
| | 83858 | ATAD3B | ATPase family, AAA domain containing 3B | Ψ 1.01 | |
| | 57130 | ATP13A1 | ATPase type 13A1 | Ψ 1.02 | |
| | 84319 | C3orf26 | Chromosome 3 open reading frame 26 | 0.99 Ω | |
| | 79913 | ACTR5 | ARP5 actin-related protein 5 homolog (yeast) | Ψ 1.01 | |
| | 79072 | FASTKD3 | FAST kinase domains 3 | 0.99 Ω | |
| | 9748 | SLK | STE20-like kinase | 0.99 Ω | |
| | 79675 | FASTKD1 | FAST kinase domains 1 | 0.99 Ω | |
| | 6596 | HLTF | Helicase-like transcription factor | 0.99 Ω | Ψ 1.01 |
| | 86 | ACTL6A | Actin-like 6A | 0.98 Ω | |
| | 79877 | DCAKD | Dephospho-CoA kinase domain containing | Ψ 1.03 | |
| | 833 | CARS | Cysteinyl-tRNA synthetase | 1.00 Ω | 0.98 Ω |
| | 79039 | DDX54 | DEAD (Asp-Glu-Ala-Asp) box polypeptide 54 | 0.99 Ω | |
| | 8491 | MAP4K3 | Mitogen-activated protein kinase kinase kinase kinase 3 | 0.98 Ω | |
| | 27347 | STK39 | Serine threonine kinase 39 | 0.98 Ω | |
| | 10730 | YME1L1 | YME1-like 1 (S. cerevisiae) | 1.00 Ω | |
| | 54802 | TRIT1 | TRNA Isopentenyltransferase 1 | 0.98 Ω | |
| Cytokine activity | 3597 | IL13RA1 | Interleukin 13 receptor, alpha 1 | Ψ 1.02 | 0.99 Ω |
| | 654 | BMP6 | Bone morphogenetic protein 6 | Ψ 1.01 | |
| | 53342 | IL17D | Interleukin 17D | 0.98 Ω | |
| Endothelial cell related | 4851 | NOTCH1 | Notch 1 | Ψ 1.02 | |
| | 889 | KRIT1 | KRIT1, ankyrin repeat containing | 0.99 Ω | |
| | 25960 | GPR124 | G protein-coupled receptor 124 | Ψ 1.02 | 0.98 Ω |
| | 2296 | FOXC1 | Forkhead box C1 | Ψ 1.01 | |
| | 29923 | HIG2 | Chromosome 7 open reading frame 68 | Ψ 1.01 | |
| | 2022 | ENG | Endoglin | Ψ 1.01 | 0.98 Ω |
| | 3728 | JUP | Junction plakoglobin | Ψ 1.01 | |
| NF-kappaB transcription | 51495 | PTPLAD1 | Protein tyrosine phosphatase-like A domain containing 1 | Ψ 1.01 | |
| | 149951 | COMMD7 | COMM domain containing 7 | 0.99 Ω | Ψ 1.01 |
| | 7159 | TP53BP2 | Tumor protein p53 binding protein, 2 | Ψ 1.01 | |
| | 54602 | NDFIP2 | Nedd4 family interacting protein 2 | 0.99 Ω | |
| Nitric oxide synthase activity | 29923 | HIG2 | Chromosome 7 open reading frame 68 | Ψ 1.01 | |
| | 2022 | ENG | Endoglin | Ψ 1.01 | 0.99 Ω |
| | 92 | ACVR2A | Activin A receptor, type IIA | 0.99 Ω | |
| Phospholipid scramblase | 57088 | PLSCR4 | Phospholipid scramblase 4 | 0.99 Ω | Ψ 1.03 |
| Actin binding | 5339 | PLEC1 | Plectin | Ψ 1.03 | |
| | 11151 | CORO1A | Coronin, actin binding protein, 1A | 0.94 Ω | |
| Heat shock protein | 55735 | DNAJC11 | DnaJ (Hsp40) homolog, subfamily C, member 11 | Ψ 1.01 | |
| | 22826 | DNAJC8 | DnaJ (Hsp40) homolog, subfamily C, member 8 | 0.99 Ω | |

TABLE 2F
Colon cancer

| Pathway | Gene 1D | Code | Name | HGERT/FIG | NG/NG |
|---|---|---|---|---|---|
| Cysteine/gluathione | 10768 | AHCYL1 | Adenosylhomocysteinase-like 1 | Ψ 1.01 | |
| | 471 | ATIC | 5-aminoimidazole-4-carboxamide ribonucleotide | 0.99 Ω | Ψ 1.01 |
| | 2098 | ESD | Esterase D | 0.99 Ω | |
| Peptidase activity | 26168 | SENP3 | SUMO1/sentrin/SMT3 specific peptidase 3 | Ψ 1.01 | |
| | 128488 | WFDC12 | WAP four-disulfide core domain 12 | 0.96 Ω | Ψ 1.01 |
| | 4287 | ATXN3 | Ataxin 3 | 0.99 Ω | |
| | 55593 | OTUD5 | OTU domain containing 5 | Ψ 1.01 | |
| Receptor activity | 152559 | PAQR3 | Progestin and adipoQ receptor family member III | 0.98 Ω | Ψ 1.01 |
| Focal adhesion | 1288 | COL4A6 | Collagen, type IV, alpha 6 | Ψ 1.03 | 0.98 Ω |
| PI3 kinase binding | 11151 | CORO1A | Coronin, actin binding protein, 1A | 0.94 Ω | |
| ATP binding and metabolism | 3984 | LIMK1 | LIM domain kinase 1 | Ψ 1.01 | |
| | 2011 | MARK2 | MAP/microtubule affinity-regulating kinase 2 | Ψ 1.03 | |
| | 6102 | RP2 | Retinitis pigmentosa 2 (X-linked recessive) | 0.98 Ω | |
| | 514 | ATP5E | ATP synthase, H+ transporting, mitochondrial F1 | 0.99 Ω | Ψ 1.01 |
| | 6196 | RPS6KA2 | Ribosomal protein S6 kinase, 90kDa, polypeptide 2 | Ψ 1.01 | 0.98 Ω |
| | 4067 | LYN | V-yes-1 Yamaguchi sarcoma viral related oncogene | Ψ 1.02 | 0.98 Ω |
| | 3336 | HSPE1 | Heat shock 10kDa protein 1 (chaperonin 10) | 0.99 Ω | |
| | 53944 | CSNK1G1 | Casein kinase 1, gamma 1 | Ψ 1.02 | |
| | 27032 | ATP2C1 | ATPase, Ca++ transporting, type 2C, member 1 | 0.99 Ω | |
| | 23095 | KIF1B | Kinesin family member 1B | Ψ 1.01 | |
| | 23456 | ABCB10 | ATP-binding cassette, sub-family B (MDR/TAP), | 0.98 Ω | Ψ 1.03 |
| | 9525 | VPS4B | Vacuolar protein sorting 4 homolog B (S. cerevisiae) | 0.99 Ω | Ψ 1.01 |
| | 84896 | ATAD1 | ATPase family, AAA domain containing 1 | 0.99 Ω | |
| | 8803 | SUCLA2 | Succinate-CoA ligase, ADP-forming, beta subunit | 0.99 Ω | Ψ 1.01 |
| | 539 | ATP5O | ATP synthase, H+ transporting, mitochondrial Fl | 1.00 Ω | |
| | 83858 | ATAD3B | ATPase family, AAA domain containing 3B | Ψ 1.01 | |
| | 57130 | ATP13A1 | ATPase type 13A1 | Ψ 1.02 | |
| | 84319 | C3orf26 | Chromosome 3 open reading frame 26 | 0.99 Ω | |
| | 79913 | ACTR5 | ARP5 actin-related protein 5 homolog (yeast) | Ψ 1.01 | |
| | 79072 | FASTKD3 | FAST kinase domains 3 | 0.99 Ω | |
| | 9748 | SLK | STE20-like kinase | 0.99 Ω | |
| | 79675 | FASTKD1 | FAST kinase domains 1 | 0.99 Ω | |
| | 6596 | HLTF | Helicase-like transcription factor | 0.99 Ω | Ψ 1.01 |
| | 86 | ACTL6A | Actin-like 6A | 0.98 Ω | |
| | 79877 | DCAKD | Dephospho-CoA kinase domain containing | Ψ 1.03 | |
| | 833 | CARS | Cysteinyl-tRNA synthetase | 1.00 Ω | 0.98 Ω |
| | 79039 | DDX54 | DEAD (Asp-Glu-Ala-Asp) box polypeptide 54 | 0.99 Ω | |
| | 8491 | MAP4K3 | Mitogen-activated protein kinase kinase kinase kinase | 0.98 Ω | |
| | 27347 | STK39 | Serine threonine kinase 39 | 0.98 Ω | |
| | 10730 | YME1L1 | YME1-like 1 (S. cerevisiae) | 1.00 Ω | |
| | 54802 | TRIT1 | TRNA isopentenyltransferase 1 | 0.98 Ω | |
| Nucleic acid binding | 24149 | ZNF318 | Zinc finger protein 318 | Ψ 1.02 | |
| | 79830 | ZMYM1 | Zinc finger, MYM-type 1 | 0.98 Ω | Ψ 1.01 |

### Example 4

Geotrichum candidum is a fungus or mold that is widely used as culture in the fermentation production of many foods including, but not limited to, dairy products such as soft cheeses like Camembert, Saint-Nectaire, Tomme de Savoie, and other fermented dairy products like yogurt and curd milk.

A starter culture with Geotrichum candidum cells (GCC) was produced by ID growing GCC in a medium containing 10% glucose and other nutrients. At the end of the fermentation process when the number of GCC did not further increase, the fermentation broth was freeze dried so that the GCC culture was preserved. The function and activity of GCC was reduced during such preservation process. The influence of ERT on GCC function and activity was studied by adding ERT to the GCC fermentation broth before freeze drying and compare function and activity of freeze-dried GCC with and without addition of ERT. Function and activity was expressed by measuring how many doses GCC culture with a standardized amount of activity and function can be obtained per liter fermentation broth.

TABLE 3
All GCC fermentation started with a glucose concentration of 10 g/ltr

| | Amount of ERT added to fermentation | | | |
|---|---|---|---|---|
| | broth before preservation | | | |
| | 0% (blank) | 2% | 3% | 5% |
| CFU/ml fermentation broth | 1.11 * 10⁸ | 1.11 * 10⁸ | 1.11 * 10⁸ | 1.11 * 10⁸ |
| Doses/ltr fermentation broth | 517 | 621 | 652 | 555 |
| Change in GCC cell activity | 100 | 120 | 126 | 107 |
| and function (blank set at | | | | |
| 100) | | | | |

Table 3 provides data illustrating that the addition of 2% and 3% ERT to the fermentation broth improved GCC activity and function by 20% and 26%, respectively. Addition of 5% ERT was apparently too high since activity and function only increased by 8%.

### Example 5

Beta cells (Cell line: HIT-T15) were obtained from supplier ATCC. The cells were seeded in 24 well plate (300,000 cells/well). After overnight attaching, the medium was removed and the cells were washed with HBSS. New medium was added, and 1/10 volume of 50 mM ERT or vehicle (medium) was added to the cells. After 1 hour incubation, glucose solution was added to a final concentration of 6, 15, or 30 mM in the wells. Subsequently, the cells were incubated for 24 hours. After incubation, all cell material including medium and HBSS was collected and centrifuged (5 minutes, 500×g). The supernatant was removed and the cells were resuspended in 500 μl HBSS. The cell suspensions were diluted 1:1 in 0.4% trypan blue dye and loaded in a Countess Chamber. The cells were counted using the Countess cell counter, which calculated the viability (function and activity) of the cells.

FIG. 6 illustrates how incubation with increasing levels of glucose resulted in a dose-dependent decrease in viability (function and activity) of HIT-T15 cells. The same incubations in the presence of 5 mM ERT attenuated this decrease significantly and resulted in an improvement in cell function and activity. The highest improvement was achieved under the highest stress caused by a 24-hour incubation with 30 mM glucose. Similar stress conditions may present as acute with healthy people and as acute to chronic with people with an impaired glucose metabolism, including, but not limited to, people who suffer from diabetes.

### Example 6

The effects of meso-1,2,3,4-tetrahydroxybutane on glucose induced changes in viability and insulin production related genes in human beta cells were studied. Beta cells are cells in the islets of Langerhans of the pancreas that secrete insulin. Human beta cells can be cultured and grown in vitro, with the 1.1E7 cell line that was developed by the University of Ulster and is commercially available at the European Collection of Cell Cultures (ECACC).

Cell culture: Human 1.1E7 beta cells were cultured in RPMI 1640 medium containing 10% fetal calf serum, 2 mM L-Glutamine and 1% penicillin/streptomycin. Cells were maintained at 37° C. in a 5% CO₂ atmosphere.

Viability: Cells were seeded in a 24 well plate. After overnight attaching, the medium was removed and cells were washed with HBSS. New medium was added and meso-1,2,3,4-tetrahydroxybutane (final concentration: 5 mM) or vehicle solution (medium) was added to the cells. After 1 hour incubation a vehicle or glucose solution was added to a final concentration of 10, 30 or 45 mM in the wells. Subsequently, cells were incubated for 24 hours, 48 hours, 6 days, and 7 days. After incubation, all cell material including medium and HBSS was collected and centrifuged (5 minutes, 500×g). Supernatant was removed and cells were resuspended in 100 μl HBSS. Cell suspensions were diluted 1:1 in 0.4% trypan blue dye and loaded in a Countess Chamber. Cells were counted using the Invitrogen Countess® automated cell counter (Life Technologies Europe BV, Bleiswijk, the Netherlands).

Gene expression analysis: RNA was isolated from Qiazol suspended cells according to the manufacturer's protocol and quantified spectrophotometrically. Reverse transcription reaction was performed using 500 ng of RNA, which was reverse-transcribed into cDNA using iScript cDNA synthesis kit. Next, real time PCR was performed with a BioRad MyiQ iCycler Single Color RT-PCR detection system using Sensimix™Plus SYBR and Fluorescein, 5 μl diluted (10×) cDNA, and 0.3 μM primers in a total volume of 25 μl. PCR was conducted as follows: denaturation at 95° C. for 10 minutes, followed by 40 cycles of 95° C. for 15 seconds and 60° C. for 45 seconds. After PCR a melt curve (60-95° C.) was produced for product identification and purity. Actin was included as internal control. Primer sequences are shown in Table 4. Data were analyzed using the MyIQ software system (Bio-Rad Laboratories BV, Veenendaal, the Netherlands) and were expressed as relative gene expression (fold change) using the 2^{ΔΔCt} method.

Table 4 below shows primer sequences for insulin production related genes used for gene expression analysis. PC1/3 and PC2 are proprotein or prohormone covertases. They convert inactive precursors of proteins (e.g. preproinsulin and proinsulin) trafficking through the secretory pathway to their mature forms (e.g. insulin). 7B2 is a small neuroendocrine protein that is required for the production of active prohormone convertase 2 (PC2)

TABLE 4

| Gene | Forward (5′ to 3′) | Reverse (5′ to 3′) |
|---|---|---|
| Human | CGGGGTACCGAAGCAAACCCAAATCTC | CTACTATTTAGCCATTTTCACCAT (SEQ |
| PC1/3 | (SEQ ID NO. 1) | ID NO 2) |
| Human | CGGGGTACCACTCTGAGGCATTCTGGG | CTACTAACATTCTTTCTTCTCAGGCAC |
| PC2 | (SEQ ID NO. 3) | GTTTC (SEQ ID NO. 4) |
| Human | TGGGCAAGTGGAACAAGAA | TGGCCAAAATAGTACAACCTCA (SEQ |
| 7B2 | (SEQ ID NO. 5) | ID NO. 6) |
| Human | CCTGGCACCCAGCACAAT (SEQ ID NO. | GCCGATCCACACGGAGTACT (SEQ ID |
| Actin | 7) | NO. 8) |

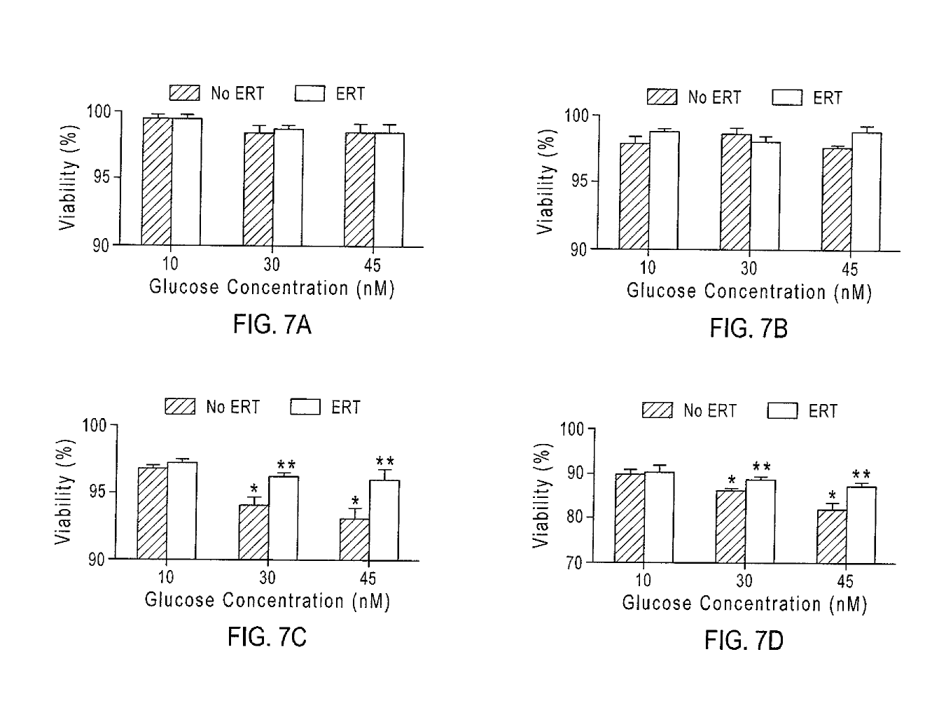

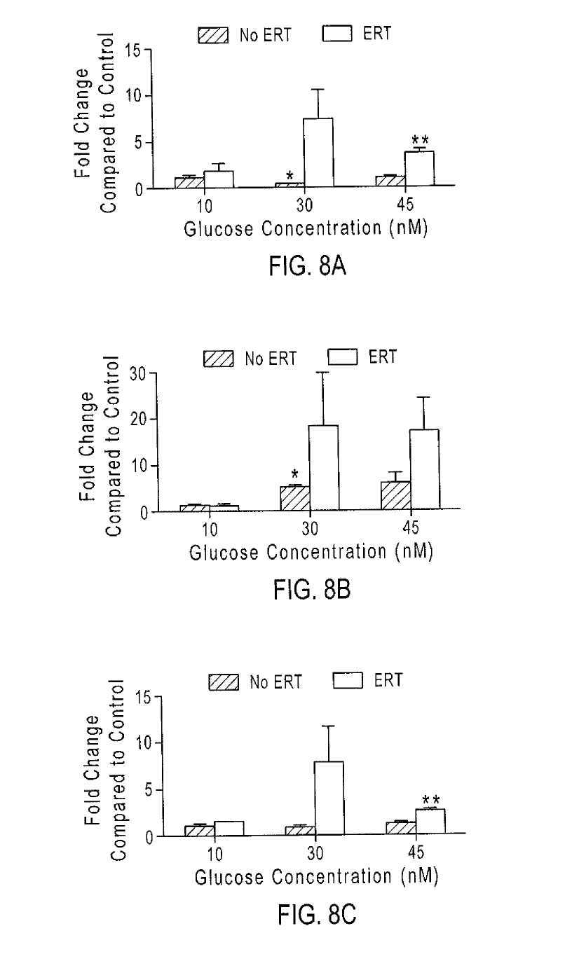

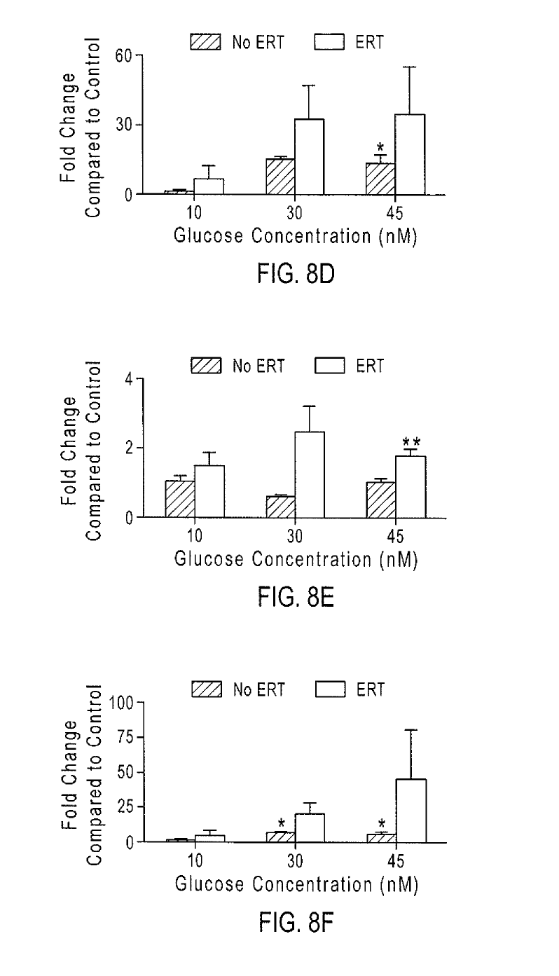

FIGS. 7A-7D are graphs showing the effect on viability of 1.1E7 cells of incubation with 10, 30 and 45 mM glucose for 24 hours (FIG. 8A), 48 hours (FIG. 8B), 6 days (FIG. 8C) or 7 days (FIG. 8D). Data are expressed as mean±standard error of four independent experiments. *=p<0.05 compared to 10 mM (normal glucose); **=p<0.05 compared to no ERT (ERT=meso-1,2,3,4-tetrahydroxybutane).

After 24 and 48 hours incubation with glucose, no impact on cell viability was observed and the presence of meso-1,2,3,4-tetrahydroxybutane did not have any impact on this maintained viability. Incubation with glucose resulted in a dose-dependent decrease in viability of 1.1E7 cells after 6 and 7 days. The presence of 5 mM of meso-1,2,3,4-tetrahydroxybutane entirely prevented this decrease in beta cell viability. The ability to maintain and/or improve the viability of beta cells during hyperglycemia may therefore be relevant and beneficial in the treatment of diabetes mellitus.

FIGS. 8A-8F are graphs showing the effect on gene expression of 7B2 (FIGS. 8A and 8B), PC1/3 (FIGS. 8C and 8D), and PC2 (FIGS. 8E and 8F) of 1.1E7 cells incubated with 10, 30 and 45 mM glucose for 24 hours (FIGS. 8A, 8C, and 8E) or 7 days (FIGS. 8B, 8D, and 8F). Data are expressed as mean±standard error of three independent experiments. *=p<0.05 compared to 10 mM (normal glucose); ** p<0.05 compared to no ERT.

Meso-1,2,3,4-tetrahydroxybutane improved the expression of genes involved in the conversion of (pre)proinsulin to insulin (See FIGS. 8A-F). These effects are especially notable after longer exposure to high concentrations of glucose. Proprotein convertase 1 (PC1) is the enzyme largely responsible for the first step in the biosynthesis of insulin. Following the action of PC1, a carboxypeptidase is required to remove the basic residues from the processing intermediate and generate the bioactive form of insulin. Another prohormone convertase, proprotein convertase 2 (PC2), plays a more minor role in the first step of insulin biosynthesis, but a greater role in the first step of glucagon biosynthesis. PC2 binds to the neuroendocrine protein named 7B2, and if this protein is not present, propC2 cannot become enzymatically active. 7B2 also inhibits PC2 activity until the 7B2 is cleaved into smaller inactive forms. Thus, 7B2 is both an activator and an inhibitor of PC2. The observation that meso-1,2,3,4-tetrahydroxybutane can increase the expression of these genes in cultured beta cells under increasing glucose concentrations and longer exposure demonstrates its ability to maintain and improve the function of human pancreatic beta cells during exposure to increasing stress conditions.

### Example 7

Meso-1,2,3,4-tetrahydroxybutane administration to Type 2 diabetes mellitus patients improved and restored glucose and insulin metabolism, thereby demonstrating maintenance and improvement of human beta cell function in vivo during exposure of diabetic stress conditions.

A human study was conducted in patients with Type 2 diabetes mellitus. Patients were enrolled who were otherwise healthy patients with Type 2 diabetes mellitus as defined by fasting blood glucose >125 mg/dl or with ongoing treatment for Type 2 diabetes mellitus with the exception of insulin.

Patients were instructed to add a dry mix sachet containing 12 g meso-n 1,2,3,4-tetrahydroxybutane to a 8 fl oz bottle of water, shake vigorously and to consume as a lemon-flavored beverage as a whole immediately.

Participants consumed meso-1,2,3,4-tetrahydroxybutane (12 g of meso-1,2,3,4-tetrahydroxybutane three times daily) for 28 days with the last consumption the evening prior to the test visit. In the morning of the test visit, fasting blood drawings were taken after which a meso-1,2,3,4-tetrahydroxybutane lemon-flavored beverage (12 g of meso-1,2,3,4-tetrahydroxybutane) together with a piece of unbuttered toast was consumed. Two hours after, blood drawings were taken again. Blood glucose, insulin and C-peptide levels were measured at both time points (i.e. before and 2 hrs after meso-1,2,3,4-tetrahydroxybutane intake).

After testing each variable for normality, a paired t-test was performed to determine statistical significance. For variables with a non-normal distribution, a Wilcoxon signed rank test was used. All analyses were performed using SPSS (IBM, version 19). Compliance was determined based on a count of empty sachets returned by study participants. Twenty-four subjects were enrolled and none withdrew from the study. Mean compliance was 90% based on a count of meso-1,2,3,4-tetrahydroxybutane sachets. Table 5 below shows the clinical characteristics of subjects at baseline.

TABLE 5
Subjects' characteristics

| Characteristics | Completed Subjects |
|---|---|
| Age (years) | 56 ± 5 |
| Females | 13 (54%) |
| Black Race | 15 (63%) |
| Weight (kg) | 90.9 ± 16.8 |
| Body mass index (kg/m2) | 30.3 ± 3.4 |
| Type 2 diabetes mellitus | 24 (100%) |
| Coronary artery disease | 5 (21%) |
| Hypertension | 20 (83%) |
| Cigarette smoking ever | 9 (38%) |
| Statin treatment | 11 (46%) |
| ACE inhibitor or ARB treatment | 13 (54%) |
| Beta blocker treatment | 6 (25%) |
| Calcium channel blocker treatment | 4 (17%) |
| Short-Acting Insulin treatment | 1 (4%) |
| Other oral diabetes treatment | 21 (88%) |
| Anti-platelet treatment | 7 (29%) |

Data are mean ± SD.
ACE = angiotensin converting enzyme;
ARB = angiotensin receptor blocker

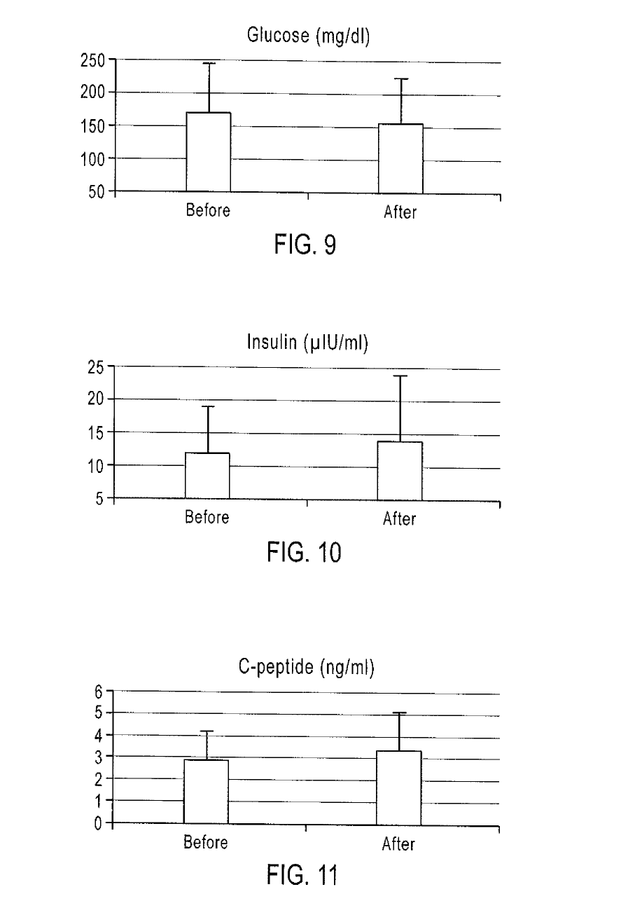

Consumption of meso-1,2,3,4-tetrahydroxybutane produced an increase in blood C-peptide levels (P=0.05), which was accompanied by a borderline significant decrease in blood glucose levels (P=0.06) and non-significant increase in insulin levels (P=0.22). FIGS. 9, 10 and 11 illustrate the changes in glucose, insulin and C-peptide before and after meso-1,2,3,4-tetrahydroxybutane intake.

Meso-1,2,3,4-tetrahydroxybutane consumption led to favourable effects on several measures of glucose metabolism, including blood glucose, insulin and C-peptide levels in Type 2 diabetes mellitus patients. This study demonstrates that meso-1,2,3,4-tetrahydroxybutane administration improved and restored glucose metabolism in patients with Type 2 Diabetes mellitus.

1. Method of maintaining or improving biological function and activity of cells over time and/or during exposure to stress, comprising using meso-1,2,3,4-tetrahydroxybutane, pure or formulated in a product or composition, whereby cells include cells from animals, humans, plants, as well as microorganisms such as bacteria, yeasts and fungi.

2. Method of increasing cell viability, comprising using meso-1,2,3,4-tetrahydroxybutane, pure or formulated in a product or composition, as a cell survival and cell protection agent, whereby cells include cells from animals, humans, plants, as well as microorganisms such as bacteria, yeasts and fungi.

3. Method of improving conversion of progenitor or stem cells to mature cells, comprising using meso-1,2,3,4-tetrahydroxybutane, pure or formulated in a product or composition, whether in vitro, in vivo, ex-vivo, or transplanted.

4. The method of claim 1 whereby cells are exposed to stress conditions caused directly or indirectly by glucose or other monosaccharides and disaccharides.

5. The method of claim 1 where cells are progenitor and mature cells lining up the inside of the vascular system.

6. The method of claim 1 where cells are progenitor and mature beta cells located in the islets of Langerhans.

7. The method of claim 1 where cells are progenitor and mature brain cells including but not limited to all cells inside the cranium or in the central spinal canal, in lymphatic tissue, in blood vessels, in the cranial nerves, in the meninges, skull, pituitary gland, or pineal gland.

8. The method of claim 7 where cells are neurons and glial cells.

9. The method of claim 8 where cells are astrocytes, oligodendrocytes, and ependymal cells.

10. The method of claim 1 where cells are microbial intestinal cells as well as progenitor and mature host intestinal cells including but not limited to cells in the colon, rectum, or in the appendix.

11. The method of claim 1 where cells are progenitor and mature prostate cells.

12. The method of claim 1 where cells are progenitor and mature breast cells including but not limited to epithelial cells from the ducti and lobules, and adipocytes.

13. The method of claim 1 where cells are microorganisms used in the fermentation step of the production process of food products chosen from fermented milk or milk derivatives, yogurt, cheese, bread, pastry, sauces, pastes, wine, beer, sauerkraut, kimchi, tempeh, and bean paste, etc.

14. The method of claim 1 where cells are microorganisms used as production strain for the manufacture of substances that are used as food, food ingredients and additives, nutraceuticals, pharmaceuticals, or personal care products.

15. The method of claim 2 whereby cells are exposed to stress conditions caused directly or indirectly by glucose or other monosaccharides and disaccharides.

16. The method of claim 2 where cells are progenitor and mature cells lining up the inside of the vascular system.

17. The method of claim 2 where cells are progenitor and mature beta cells located in the islets of Langerhans.

18. The method of claim 3 whereby cells are exposed to stress conditions caused directly or indirectly by glucose or other monosaccharides and disaccharides.

19. The method of 3 where cells are progenitor and mature cells lining up the inside of the vascular system.

20. The method of claim 3 where cells are progenitor and mature beta cells located in the islets of Langerhans.


SEQUENCE LISTING (8 sequences)

SEQ ID NO: 1 - DNA, 27 bp, Homo sapiens
cggggtaccgaagcaaacccaaatctc

SEQ ID NO: 2 - DNA, 24 bp, Homo sapiens
ctactatttagccattttcaccat

SEQ ID NO: 3 - DNA, 27 bp, Homo sapiens
cggggtaccactctgaggcattctggg

SEQ ID NO: 4 - DNA, 32 bp, Homo sapiens
ctactaacattctttcttctcaggcacgtttc

SEQ ID NO: 5 - DNA, 19 bp, Homo sapiens
tgggcaagtggaacaagaa

SEQ ID NO: 6 - DNA, 22 bp, Homo sapiens
tggccaaaatagtacaacctca

SEQ ID NO: 7 - DNA, 18 bp, Homo sapiens
cctggcacccagcacaat

SEQ ID NO: 8 - DNA, 20 bp, Homo sapiens
gccgatccacacggagtact